(19) United States
(12) Patent Application Publication
(10) Pub. No.: US 2017/0183664 A1
(43) Pub. Date: Jun. 29, 2017

(54) SMALL RNAs (sRNA) THAT ACTIVATE TRANSCRIPTION

(71) Applicant: CORNELL UNIVERSITY, Ithaca, NY (US)

(72) Inventors: Julius B. LUCKS, Evanston, IL (US); James CHAPPELL, Chicago, IL (US); Melissa TAKAHASHI, Somerville, MA (US)

(21) Appl. No.: 15/304,903

(22) PCT Filed: Apr. 16, 2015
(86) PCT No.: PCT/US15/26145

§ 371 Date: Oct. 18, 2016

Related U.S. Application Data
(60) Provisional application No. 61/981,241, filed on Apr. 18, 2014.

(51) Int. Cl. C12N 15/63; C12N 15/113; C12P 21/00

(52) U.S. Cl. CPC C12N 15/635; C12P 21/00; C12N 15/113; C12N 2310/11

(57) ABSTRACT

Disclosed herein are compositions for transcriptional regulation of a target gene, and methods of using the compositions. The compositions utilize a novel antisense RNA design that activates transcription of a target gene and involve a sense genetic construct that represses transcription of the target gene, and an antisense construct selected from an antisense activating RNA, or an antisense genetic construct encoding an antisense activating RNA, that binds to the sense genetic construct RNA to relieve repression of the target gene, thus activating expression of the gene.

### CROSS-REFERENCE TO RELATED APPLICATIONS

This application claims priority to U.S. provisional application 61/981,241, filed Apr. 18, 2014, which is incorporated herein in its entirety.

### STATEMENT REGARDING FEDERALLY SPONSORED RESEARCH OR DEVELOPMENT

This invention was made with government support under grant no. DGE-1144153 awarded by National Science Foundation; grant no. N66001-12-1-4254 awarded by the U.S. National Department of Defense, and grant no. N00014-13-1-0531 awarded by the U.S. Navy. The government has certain rights in the invention.

### INCORPORATION BY REFERENCE OF SEQUENCE LISTING

The Sequence Listing in the ASCII text file, named as 31000_663_02_SEQ.txt of 54000 bytes, created on Apr. 8, 2015, and submitted to the United States Patent and Trademark Office via EFS-Web, is incorporated herein by reference.

### BACKGROUND OF THE DISCLOSURE

RNA regulators have become an important component of the synthetic biology toolbox for controlling gene expression and constructing synthetic gene networks (Chappell, J. et al., Biotechnol. 8, 1379-1395 (2013)). They are increasingly attractive substrates owing to their mechanistic diversity and to the emergence of computational and experimental tools (Carothers, J. M., et al., Science 334, 1716-1719 (2011), Rodrigo, G., et al., Proc. Natl. Acad. Sci. USA 109, 15271-15276 (2012), Wachsmuth, M., et al., Nucleic Acids Res. 41, 2541-2551 (2013), Xayaphoummine, A., et al., Nucleic Acids Res. 35, 614-622 (2007); Lucks, J. B. et al., Proc. Natl. Acad. Sci. USA 108, 11063-11068 (2011), Rouskin, S., et al., Nature 505, 701-705 (2014)) that predict and characterize RNA structures, ultimately informing their functional design.

One of the advantages of RNA regulators over their protein counterparts is the wealth of available computational structure prediction tools that can serve as a starting point for model-guided RNA regulator design. Recently, these tools have been combined with mechanistic models of RNA regulation to rationally design and optimize a range of systems that control translation, including RBSs (Salis, H. M., et al., Nat. Biotechnol. 27, 946-950 (2009)), sRNAs (Mutalik, V. K., et al., Nat. Chem. Biol. 8, 447-454 (2012), Green, A. A., et al., Cell 159, 925-939 (2014)) and riboswitches (Wachsmuth, M., et al., Nucleic Acids Res. 41, 2541-2551 (2013)).

RNA-mediated control of gene expression often involves the formation of particular structures within mRNAs. These structures can regulate gene expression in cis, for example, by preventing transcription elongation in the case of intrinsic terminator hairpins, or by preventing translation initiation by occluding ribosome binding sites. Moreover, the formation of these cis-acting structures can also be regulated by interactions with trans-acting RNAs, creating genetic switches that are flipped at the RNA level. Although RNA structures are highly designable, being largely determined by Watson-Crick base-pairing of the four letter nucleotide code, the design of high performing synthetic RNA regulators has historically been challenging.

sRNAs that activate or repress translation are found throughout nature (Storz, G., et al., Mol. Cell 43, 880-891 (2011)) and have been engineered to tune gene expression in metabolic pathways (Na, D. et al., Nat. Biotechnol. 31, 170-174 (2013)), to silence endogenous genes in E. coli (Sharma, V., et al., ACS Synth. Biol. 1, 6-13 (2012)) and to act as key components of genetic circuits that perform cellular computations, including genetic switchboards (Callura, J. M., et al., Proc. Natl. Acad. Sci. USA 109, 5850-5855 (2012)) and counters (Friedland, A. E. et al., Science 324, 1199-1202 (2009), Isaacs, F. J. et al., Nat. Biotechnol. 22, 841-847 (2004)). Moreover, sRNAs that repress transcription have been engineered to create orthogonal and composable regulators that can be used to construct RNA-only transcriptional networks (Lucks, J. B., et al., Proc. Natl. Acad. Sci. USA 108, 8617-8622 (2011), Takahashi, M. K., et al., Nucleic Acids Res. 41, 7577-7588 (2013)). These versatile sRNA transcriptional repressors called attenuators have been used to construct RNA-only networks that can act as genetic logic gates, propagate information in transcriptional cascades and control the timing of expression of multiple genes (Lucks, J. B., et al., Proc. Natl. Acad. Sci. USA 108, 8617-8622 (2011). Furthermore, because these networks propagate signals directly as RNA species, they operate on the fast timescales set by RNA degradation rates (Takahashi, M. K. et al., ACS Synth. Biol. (available online 12 Mar. 2014)).

Bacterial attenuator sequences, such as the staphylococcal plasmid pT181 attenuator, regulate transcription elongation through RNA structural rearrangements that form an intrinsic transcription terminator hairpin upstream of the coding region (Brantl, S., et al., Mol. Microbiol. 35, 1469-1482 (2000)). For the pT181 attenuator, in the absence of antisense sRNA that binds the nascent RNA during transcription of the attenuator sequence, the attenuator RNA folds so that an anti-terminator sequence sequesters the 5′ side of the intrinsic terminator hairpin, thereby inhibiting formation of the terminator hairpin and allowing transcription elongation. When antisense sRNA is present, the antisense sRNA interacts with the attenuator region and sequesters the anti-terminator sequence, which enables terminator formation that causes RNA polymerase (RNAP) to abort transcription of the mRNA. Transcriptional attenuators such as the pT181 attenuator thus structurally encode their own repressive regulation. The pT181 attenuator in particular has been used in a number of synthetic biology applications, including the creation of a genetic network that controls the sequential timing of expression of two different genes, which could be useful in controlling the expression of metabolic enzymes (Takahashi, M. K. et al., ACS Synth. Biol.).

RNA antisense nucleic acids that activate, rather than repress, transcription of a gene of interest, would open up valuable new avenues for RNA-only regulation of target genes.

### BRIEF SUMMARY OF THE DISCLOSURE

The present invention is directed to sRNA molecules capable of activating the transcription of a target gene in vivo.

Accordingly, disclosed herein are compositions for transcriptional regulation of a target gene. The compositions include a sense genetic construct, and an antisense construct.

The sense genetic construct includes, from 5′ to 3′: a promoter sequence; a terminator sequence encoding a ribonucleic acid (RNA) terminator stem-loop, the terminator sequence including a 5′ terminator stem sequence and a 3′ terminator stem sequence that are substantially complementary to each other; and a sequence encoding a poly-uracil RNA sequence immediately 3′ of the terminator sequence. In some embodiments, the sense genetic construct has an intervening sequence between the promoter and the terminator sequence.

The antisense construct can be (i) an antisense activating RNA with substantial complementary to at least a portion of the 5′ terminator stem sequence of the sense RNA, or (ii) an antisense genetic construct encoding an antisense activating RNA, the antisense genetic construct including, from 5′ to 3′: a promoter sequence and a sequence encoding an antisense activating RNA with substantial complementary to at least a portion of the 5′ terminator stem sequence of the sense RNA. In some embodiments, the antisense genetic construct has a transcription termination sequence 3′ to the sequence encoding the antisense activating RNA.

In some embodiments, the terminator sequence of the sense genetic construct can be 10 to 300 nucleotides, 12 to 200 nucleotides, 15-150 nucleotides, or 15-100 nucleotides in length. In some embodiments, the sense terminator sequence has at least 85%, at least 90%, at least 93%, at least 95%, at least 98%, or 100% identity to a nucleic acid sequence selected from SEQ ID NOS: 1-41. In some embodiments, the 5′ terminator stem sequence has a length of 4 to 40 nucleotides, 5 to 36 nucleotides, 6 to 32 nucleotides, 7 to 28 nucleotides, 8 to 24 nucleotides, 9 to 20 nucleotides, 5 to 15 nucleotides, 40 to 80 nucleotides, or 10 to 16 nucleotides. In some embodiments, the 5′ terminator stem sequence has a guanine-cytosine (G-C) content of at least 50%, at least 60%, at least 70%, at least 80%, or at least 90%. In some embodiments, the loop between the 5′ and 3′ stem sequences has a length of 3 to 30 nucleotides, or 4 to 26 nucleotides, or 5 to 22 nucleotides, or 6 to 18 nucleotides, or 7 to 14 nucleotides, or 8 to 12 nucleotides.

In some embodiments, the poly-uracil sequence of the sense genetic construct is at least 50%, at least 60%, at least 70%, at least 80%, at least 90%, or 100% uracils. In some embodiments, the poly-uracil sequence of the sense genetic construct is 3-18, 4-15, 5-12, or 6-9 nucleotides in length.

In some embodiments, the sense genetic construct includes a constitutive, inducible, or tissue-specific promoter. In some embodiments, the sense genetic construct does not contain a sequence between the promoter and the 5′ terminator stem sequence with substantial complementarity to the 5′ terminator stem sequence. In some embodiments, the sense genetic construct has at least two, or two or more, terminator sequences in tandem. Compositions with at least two terminator sequences can also have at least two antisense constructs.

In some embodiments, the antisense activating RNA is 5 to 300 nucleotides, 6 to 200 nucleotides, 7 to 150 nucleotides, 8 to 100 nucleotides, or 10 to 50 nucleotides in length. In some embodiments, the antisense sequence has at least 85%, at least 90%, at least 93%, at least 95%, at least 98%, or 100% identity to a nucleic acid sequence selected from SEQ ID NOS: 42-87.

In some embodiments, the antisense construct of the disclosed compositions is an antisense genetic construct encoding an antisense activating RNA. Within these embodiments, the antisense genetic construct can have a transcriptional termination sequence after the antisense activating RNA sequence. In some embodiments, the antisense genetic construct includes a constitutive, inducible, or tissue-specific promoter. In some embodiments, the sense genetic construct and the antisense genetic construct have different promoters, while in other embodiments, the sense and antisense genetic constructs have the same promoter.

In addition to the above, compositions disclosed herein can further include an RNA polymerase from bacterial or bacteriophage systems, such as a T7, T5, or T3 bacteriophage polymerase, SP6 bacteriophage polymerase, U6 bacteriophage polymerase, H1 human polymerase, or Bst bacterial polymerase. In one example, the polymerase is T7 polymerase.

Further disclosed herein are methods of regulating expression of a gene of interest, involving placing the gene of interest under control of the compositions disclosed herein. In one embodiment, the sense genetic construct is inserted into a genomic sequence upstream of a gene of interest. In another embodiment, in the absence of the antisense activating RNA, transcription of the gene of interest is repressed. In a further embodiment, the antisense activating RNA activates transcription of the gene of interest. The methods can involve introducing the disclosed compositions into a prokaryotic cell, or introduced into a eukaryotic cell in vitro. The gene of interest can be endogenous or non-endogenous to the cell. The methods can be used to engineer cells for an industrial fermentation, biofuel production, or recombinant protein production process. In some embodiments, the sense genetic construct mimics a riboswitch, aptazyme, or other nucleic acid sequence that can be recognized by antisense repressor RNA molecules.

Further disclosed herein are methods of increasing the transcription of a target gene, comprising introducing the disclosed compositions into a host cell so that the sense genetic construct is in operable linkage with the target gene, and expression of the antisense activating construct increases transcription of said target gene. In some embodiments, the host cell is a eukaryotic cell.

Further disclosed herein are in vitro transcription-translation (TX/TL) systems for diagnostic or biosensor use. The disclosed TX/TL systems include a cell extract, such as an E. coli cell extract derived from a cell lysate, a solution with components for transcription and translation, and a DNA template.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1E. Design and characterization of the direct anti-terminator STAR mechanism. (A) Schematic of the mechanism. In the absence of a STAR antisense, an intrinsic terminator is formed in the sense target RNA preventing transcription elongation (OFF). In the presence of the STAR antisense, the 5′ intrinsic terminator stem is sequestered by the STAR antisense, allowing downstream transcription by RNAP. This mechanism removes a structural repression connection from the attenuation mechanism inverting the function from repression to activation, as shown at the bottom. (B-D) Fluorescence characterization was performed (measured in units of fluorescence (FL)/optical density (OD) at 600 nm) on STAR sense targets (S) in the absence of STAR antisense (−A) and presence of STAR antisense (+A) for the T181 (B), AD1 (C) and pbuE (D) systems. Fold activations are labeled above each A/S pair tested. In B, +A variants are color-coded according to sequence optimizations. Data represent mean values of n=9 biological replicas±s.d. (E) Comparison of qPCR and fluorescence characterization of the best STAR-target variants. Fluorescence data are from panels B-D. The ON condition for the qPCR and FL/OD data were normalized to 1 within each system. qPCR data represent mean values of n=3 biological replicas±s.d. For both qPCR and FL/OD data, a Welch's t-test was performed on each −A/+A pair; *P<0.05, indicating conditions where the FL/OD for the +A condition was statistically significant from that of the −A condition.

FIG. 2. Schematic of the optimization of the T181 direct anti-terminator STARs. The natural pT181 attenuator is represented by the colored bar with nucleotide scale below. The optimizations of the sense target RNAs (forward arrows above bar) are aligned to the natural pT181 sequence. The optimization of the STAR antisense (reverse arrows below bar) are aligned to the region of complementarity. Arrows indicate the 3′ end of RNAs, and crosses indicate a region of non-complementarity. STAR antisense lengths do not include the t500 transcription terminator present in their expression context.

FIGS. 3A-3C. Characterization of additional anti-terminator STARs. STARs were constructed to target intrinsic terminators from (A) transcriptional attenuators and (B) transcriptional riboswitches, and fluorescence characterization performed (measured in units of fluorescence [FL]/optical density [OD] at 600 nm) on different sense targets (S) in the absence of STAR antisense (−A) and presence of STAR antisense (+A). Data represents mean values of n=9±biological replicas standard deviation. Fold activations are labeled above each A/S pair tested. Welch's t-test was performed on each −A/+A pair; * indicates conditions where the FL/OD for the +A condition was statistically significant from the −A condition (p<0.05). (C), Optimization of the pbuE direct anti-terminator STARs. STAR antisense and sense target RNAs were lengthened in 10 nucleotide increments and fluorescence characterization performed (measured in units of fluorescence [FL]/optical density [OD] at 600 nm) on different sense targets (S) in the absence of STAR antisense (−A) and presence of STAR antisense (+A). Data represents mean values of n=9 biological replicas±standard deviation. Fold activations are labeled above each A/S pair tested. Welch's t-test was performed on each −A/+A pair; * indicates conditions where the FL/OD for the +A condition was statistically significant from the −A condition (p<0.05).

FIGS. 4A-4B. Characterization of STARS using in vitro transcription and translation (TX-TL) reactions. Fluorescence characterization in TX-TL of the (A) AD1 and (B) T181 STARs. Data represents mean values of n=3 biological replicas±standard deviation.

FIGS. 5A-5E. STAR design principles. (A) A kinetic model of STAR anti-termination showing the hypothesized interactions between the STAR antisense and the sense target region. This model considers an initial state and a SEED complex (SC). The initial state consists of a fully transcribed STAR antisense, with free energy ΔG_{STAR}, and the upstream portion of the sense target that is transcribed before the transcription elongation decision has been made, with free energy ΔG_{Target}. These interact with a forward rate k_{f} to form the SC with free energy ΔG_{SC}. Under the hypothesis that the formation of the SC is sufficient to allow transcription elongation and downstream gene expression, the natural log of observed gene expression (fluorescence (FL)/optical density (OD)) is linearly related to ΔG_{prediction}, which is the difference in free energies between the initial state and SC. (B-E) Observed correlations between fluorescence characterization (measured in units of natural log FL/OD at 600 nm) and ΔG_{prediction} of different length STARs against the optimally functioning target region from the T181 (B), AD1 (C), pbuE (D) systems (shown in FIG. 1E) and the intrinsic terminator of the E. coli ribA gene (E). Data represent mean values of n=9 biological replicas±s.d. The R² correlation coefficient between ln(FL/OD) and ΔG_{prediction} is shown in the upper left of each plot.

FIG. 6. Characterization of different combinations of STAR antisense and sense target lengths. A matrix of all STAR antisense and five sense target length combinations was characterized for the T181, AD1 and pbuE systems. For each combination of STAR antisense/sense target plasmids fluorescence characterization (measured in units of fluorescence [FL]/optical density [OD] at 600 nm) was performed. For each sense target, all STAR antisense combinations were normalized by dividing the FL/OD of each antisense by the highest observed FL/OD for that sense target. STAR antisense lengths include the t500 transcription terminator. Data represents mean values of n=3 biological replicas±standard deviation. Length of STAR antisense variants are plotted on the x-axis and the sense target length is indicated above each plot.

FIGS. 7A-7B. Characterization and optimization of STAR antisenses designed to target intrinsic terminators from the E. coli genome. (A) Characterization of four sense target variants whereby intrinsic terminators from endogenous genes were placed upstream of a strong RBS and SFGFP in our two-plasmid system. Complementary STAR antisenses were designed to target the 5′ half of the terminator. (B) Characterization of STAR antisense length variants targeting the ribA terminator. Fluorescence characterization was performed (measured in units of fluorescence [FL]/optical density [OD] at 600 nm) on different sense targets (S) in the absence of STAR antisense (−A) and presence of STAR antisense (+A). Data represents mean values of n=9 biological replicas±standard deviation. Fold activations are labeled above each A/S pair tested. Welch's t-test was performed on each −A/+A pair; * indicates conditions where the FL/OD for the +A condition was statistically significant from the −A condition (p<0.05).

FIG. 8. Determining the orthogonality of STAR regulators and transcriptional attenuators. Characterization of an 8×8 orthogonality matrix of four different STAR regulators and four transcriptional repressors. Each element of the matrix represents the fold change of gene expression for the indicated antisense/sense target plasmid combination compared to a no-antisense/sense target plasmid condition. Fold changes for different combinations are written within each of the elements of the matrix. Fluorescence characterization (measured in units of fluorescence/optical density at 600 nm) was used to calculate fold change, which is represented by a color scale in which values ≧tenfold are blue (activation), onefold is white (no activation, no repression) and negative fivefold are red (repression). Data represents mean values of n=9 biological replicas±s.d.

FIGS. 9A-9B. Characterization of novel RNA-only transcriptional logic gates. (A,B) DNA template (upper left), logic schematic (lower left) and fluorescence characterization (measured in units of fluorescence (FL)/optical density (OD) at 600 nm) (right) of the A AND B logic gate (A) and the A AND NOT B logic (B) gate. Fluorescence data were normalized to 1 for the ON condition in the presence of both antisenses A and B in a or in the presence of only antisense A in b. Insets show the measured output (normalized FL/OD measurements) performances of the logic gates and the expected values for a perfect digital logic gate (parentheses). Data represent mean values of n=9 biological replicas±s.d. Welch's t-test was performed on each ON/OFF condition; *P<0.05, indicating conditions where the FL/OD for the ON condition was statistically significant from all OFF conditions.

FIGS. 10A-10B. STAR regulation applied to improve existing technologies. (A), STAR sense/antisense regulation of enzymatic pathways can be used to improve metabolic pathways for enzyme expression and strain engineering. (B), STAR sense/antisense technology can be used as a biosensor in transcription/translation (TX-TL) diagnostic assays. In these assays, the STAR antisense molecule is designed to bind a nucleic acid to be detected and the sense genetic construct is placed upstream of a reporter molecule. The presence of the nucleic acid of interest in a sample alters the conformation of the antisense RNA from an inactive to an active form. The active antisense RNA binds to the sense sequence and activates transcription of the reporter gene, allowing detection of the nucleic acid of interest.

FIG. 11. Sense/Antisense sequences validate design principles. Sense/antisense sequences designed according to the disclosed methods function to activate transcription. Sense target 1, SEQ ID NO: 105. STAR Antisense 1, SEQ ID NO: 110. Sense target 2, SEQ ID NO: 106. STAR Antisense 2, SEQ ID NO: 111. Sense target 3, SEQ ID NO: 107. STAR Antisense 3, SEQ ID NO: 112. Sense target 4, SEQ ID NO: 108. STAR Antisense 4, SEQ ID NO: 113. Sense target 5, SEQ ID NO: 109. STAR Antisense 5, SEQ ID NO: 114. Each combination of STAR/sense sequence provides significantly increased activation of transcription of the target gene, thus validating the design principles.

### DETAILED DESCRIPTION OF THE DISCLOSURE

Disclosed herein are sRNA-mediated transcriptional activators that function through a trans-acting anti-terminator mechanism to regulate transcription of a target gene.

### Compositions for Transcriptional Regulation

Compositions disclosed herein include a sense genetic construct encoding a molecule that represses transcription of a target gene, and an antisense construct that alleviates repression of the target gene by the sense construct, leading to activation or increased expression of the target gene.

The terms “target gene” and “gene of interest” are used interchangeably throughout this disclosure and encompass any gene for which regulation is desired. The target gene or gene of interest may encode, for example, an endogenous, exogenous, or recombinant protein.

### Sense Genetic Construct

The sense genetic construct includes, from 5′ to 3′: a promoter sequence; a terminator sequence encoding a ribonucleic acid (RNA) terminator stem-loop, said terminator sequence comprising a 5′ terminator stem sequence and a 3′ terminator stem sequence that are substantially complementary to each other; and a sequence encoding a poly-uracil (poly-U) RNA sequence immediately 3′ of the terminator sequence.

Part of the terminator sequence of the sense construct encodes an RNA hairpin that folds on itself to form a stem-loop with 5′ and 3′ stem ends. The term “encodes” refers to a nucleic acid sequence which codes for a polypeptide sequence or for a non-translated RNA, such as a regulatory RNA, antisense RNA, or other small RNA. A “hairpin” structure is a nucleic acid molecule that partially anneals to itself to form a secondary structure that includes a single-stranded “loop” domain that is not substantially complementary to another portion of the hairpin, and a double-stranded stem domain composed of substantially complementary sequences in the nucleic acid molecule that anneal to each other. The “stem” sequence is thus the portion of the terminator sequence that binds its complementary strand on the terminator sequence but does not include the “loop” portion of the terminator. The 5′ terminator stem sequence and the 3′ terminator stem sequence are substantially complementary to each other, such that as transcription occurs, the 5′ and 3′ RNA sequences anneal to one another to form the stem of the hairpin.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes, including the wobble base pair formed between U and G. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Two sequences are “substantially complementary” when the sequences anneal with one another under appropriate conditions, such as inside a host cell under temperature and environmental conditions that are suitable for the cell, or under stringent annealing conditions outside of a host cell. By “substantially complementary” is also meant that two sequences have at least about 70%, 75%, 80%, 85%, 90%, 95%, 96%, 97%, 98%, or 99% identity to one another, or have 100% identity to each other, across at least a portion of the sequence.

The term “stringent annealing conditions” is defined as conditions under which a nucleotide sequence anneals specifically with a target sequence(s) and not with non-target sequences, as can be determined empirically. The term “stringent conditions” is functionally defined with regard to the annealing of a nucleic-acid primer to a target nucleic acid (i.e., to a particular nucleic acid sequence of interest) by the specific annealing procedures discussed in Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

The 5′ terminator stem sequence and the 3′ terminator sequence bind together to form a terminator stem-loop formation in the transcribed sense RNA, upstream of the target gene. As the RNA polymerase transcribes the polyU sequence, the polymerase pauses, and formation of the terminator stem behind the RNA polymerase during this pause causes the polymerase to abort transcription and separate from the DNA sequence. This terminates transcription of the sense RNA prior to transcription of the target gene, thus preventing transcription of the target gene.

In some embodiments, the 5′ terminator stem sequence has a length of at least 4, at least 5, at least 6, at least 7, at least 8, at least 9, or at least 10 nucleotides, and not more than 15, 20, 25, 30, 35, or 40 nucleotides. For example, the 5′ terminator stem sequence can have a length in the range of 4 to 40 nucleotides, 5 to 35 nucleotides, 6 to 30 nucleotides, 7 to 25 nucleotides, 8 to 20 nucleotides, or 10 to 15 nucleotides. In some embodiments, the 5′ and 3′ stem sequences are equal in length, while in other embodiments the 3′ stem is longer or shorter than the 5′ stem by one or more nucleotides. In some embodiments, the 5′ terminator stem sequence has a guanine-cytosine (G-C) content of 30-60%, 30-100%, or at least 30%, at least 40%, at least 50%, at least 60%, at least 70%, at least 80%, or at least 90%. See, as non-limiting examples, the 5′ terminator stem portion of SEQ ID NO: 107, residues 19-36 (a 5′ stem of 18 nucleotides in length with 44% G-C content); and the 5′ terminator stem portion of SEQ ID NO: 108, residues 18-36 (a 5′ stem of 19 nucleotides in length with 32% G-C content). In some embodiments, the loop between the 5′ and 3′ stem sequences has a minimum length of about 3, 4, 5, 6, 7, or 8 nucleotides, and a maximum length of about 12, 14, 16, 18, 20, 22, 24, 26, 28, or 30 nucleotides. For example, the loop sequence can have a range of about 3 to 30 nucleotides, or 4 to 26 nucleotides, or 5 to 22 nucleotides, or 6 to 18 nucleotides, or 7 to 14 nucleotides, or 8 to 12 nucleotides.

The genetic constructs disclosed herein, including sense genetic constructs and antisense activating genetic constructs, also preferably include a promoter to initiate transcription of the sense or antisense RNA, as appropriate.

As used herein, a “promoter” refers to a DNA sequence recognized by the molecular machinery of the cell, or introduced molecular machinery, required to initiate the transcription of a genetic locus. The promoter sequence can be a constitutive or inducible promoter.

In some embodiments, the promoter is a constitutive promoter. A constitutive promoter is an unregulated promoter that allows for continual transcription. Constitutive bacterial promoters include, for example, the family of E. coli constitutive promoter “parts” J23100 through J23119 listed in the Registry of Standard Biological Parts on the website of the International Genetically Engineered Machine (iGEM) Foundation. A modified J23119 promoter sequence that approximates the consensus sequence for this family of promoters is the sequence TTGACAGCTAGCTCAGTCCTAGGTATAATACTAGT (SEQ ID NO: 92). Additional examples of constitutive promoters in prokaryotes include B. subtilis veg, ctc, gsi, and 43 promoters; and T7 phage promoters. In E. coli, one consensus constitutive promoter sequence includes a pair of hexanucleotide sequence elements, TTGACA and TATAAT, which are situated at 10 and 35 bp upstream, respectively, of a transcription initiation site, with a spacer DNA of approximately 17 bp separating these two sequence elements. See, Shimada T. et al., PloS ONE 9(6): e100908 (2014) for review and list of constitutive promoters in E. coli that are suitable for use with the methods disclosed herein. Constitutive yeast promoters include ADH, pCYC, or LEU2. Constitutive mammalian promoters include the human EF1-alpha elongation factor promoter, CMV (cytomegalovirus) immediate early promoter and CAG chicken albumin promoter.

In some embodiments, the promoter is an inducible promoter that allows one to control transcription of the sense and/or antisense RNA. Suitable examples of inducible promoters include tetracycline-regulated promoters (tet on or tet off) and steroid-regulated promoters derived from glucocorticoid or estrogen receptors. Examples of inducible prokaryotic promoters include the major right and left promoters of bacteriophage (P.sub.L and P.sub.R), the trp, recA, lacZ, AraC and gal promoters of E. coli, the alpha-amylase (amyE) and the sigma-28-specific promoters of B. subtilis, the promoters of the bacteriophages of Bacillus, and the like. Inducible yeast promoters include HIS3, PGK, PHOS, GAPDH, ADC1, TRP1, URA3, ENO, TPI, and AOX1. Inducible mammalian promoters include, for example, hormone-inducible promoters. Alternatively, the promoter can be a promoter that is activated in specific cell types and/or at particular points in development.

The sense genetic construct encodes a poly-uracil/poly-U sequence immediately 3′ of the terminator sequence. By “poly-uracil” is meant that the poly-uracil sequence is at least 50%, at least 60%, at least 70%, at least 80%, at least 90%, or 100% uracils. By “immediately 3′ of the terminator sequence” is generally meant that the poly-U sequence commences at the next 3′ nucleotide after the last nucleotide of the 3′ stem sequence that is complementary to the 5′ stem sequence. However, in some embodiments, “immediately 3′ of the terminator sequence” can mean that the poly-U sequence is separated from the 3′ stem by up to 1, up to 2, up to 3, up to 4, up to 5, or up to 6 nucleotides. In some embodiments, the poly-uracil sequence has a length of at least 3, at least 4, at least 5, or at least 6, and not more than 9, 12, 15, or 18 nucleotides, for example, the poly-uracil sequence can have a length in the range of 3-18, 4-15, 5-12, or 6-9 nucleotides.

The sense construct can include a sequence between the promoter and the 5′ stem sequence. This “intervening” sequence between the promoter and the 5′ stem sequence can be 0, 1, 2, 3, 4, 5, 6, 7, 8, 9, 10, or nucleotides, but not more than 50, 100, 150, 200, 250, or 300 nucleotides. Thus the intervening sequence can be 0-300 nucleotides, 2-250 nucleotides, 4-200 nucleotides, 6-150 nucleotides, 8-100 nucleotides, or 10-50 nucleotides in length. In some embodiments, the sequence between the promoter and the 5′ stem sequence has no sequence that is substantially complementary to the 5′ stem sequence. In these embodiments, the absence of complementary sequence between the promoter and the 5′ stem sequence provides for formation of the terminator stem-loop and prevention of target gene transcription “by default”, that is, in the absence of antisense RNA activation. In other embodiments, the intervening sequence can include an “interaction sequence” that the antisense RNA can bind to. In this embodiment, the antisense RNA can bind to both the interaction sequence and the 5′ terminator stem sequence in the transcribed sense RNA.

In some embodiments, the length of the terminator sequence, from the first nucleotide 3′ to the promoter sequence to the 3′ end of the poly-U sequence, including any sequence from the promoter to the 5′ stem, the 5′ stem sequence, the loop sequence, the 3′ stem sequence, and the poly-U tail, is at least 10, at least 12, at least 15, or at least 20 nucleotides in length, but not more than 100, 150, 200, 250, or 300 nucleotides in length. For example, the terminator sequence can have a length in the range of 10 to 300 nucleotides, 12 to 200 nucleotides, 15-150 nucleotides, or 20-100 nucleotides. In some embodiments, the sense terminator sequence has at least 85%, at least 90%, at least 93%, at least 95%, at least 98%, or 100% identity to a nucleic acid sequence selected from SEQ ID NOS: 1-41 and 105-109, or SEQ ID NOS: 1-37 and 105-109. In other embodiments, the terminator sequence can be about 336, 322, 320, 319, 293, 292, 287, 259, 231, 208, 198, 188, 178, 168, 163, 158, 152, 148, 139, 135, 120, 113, 110, 103, 100, 93, 90, 83, 80, 73, 63, 59, 58, 56, 52, or 43 nucleotides in length. Specific examples of sense sequences are listed in Table 2.

The term “about” as used throughout this application is defined to be within 10%, within 5%, within 2%, or within 1% of the numbered value.

In other embodiments, the terminator sequence is derived from, that is, designed based upon, a bacterial transcriptional attenuator sequence. Examples of transcriptional attenuators include pT181, pIP501, pCF10 and pAD1.

### Activating Antisense Nucleic Acid

Further disclosed herein are antisense constructs. The construct can be an antisense activating RNA, or a genetic construct (the “antisense genetic construct”) encoding an antisense activating RNA. The terms “antisense activating RNA” and “small transcription activating RNA” or “STAR” are used interchangeably throughout this disclosure.

An antisense activating RNA is an RNA with substantial complementary to at least a portion of the 5′ terminator stem sequence of the sense RNA. By “at least a portion” of the 5′ terminator stem is meant that the antisense activating RNA binds to the 5′ terminator stem sequence sufficiently to prevent binding of the 5′ stem and 3′ stem and prevent formation of the terminator stem-loop. By “at least a portion” is also meant that the antisense RNA is complementary to the 5′ stem at at least 3, at least 4, at least 5, at least 6, at least 7, at least 8, at least 9, at least 10, at least 12, at least 15, at least 20, or at least 25 nucleotides of the 5′ stem sequence, or that the antisense RNA anneals to at least 50%, at least 60%, at least 70%, at least 80%, at least 90%, at least 95%, or 100% of the length of the 5′ stem sequence. The antisense RNA activates transcription of a target gene by binding to the 5′ terminator stem sequence of the sense RNA, which prevents binding of the target RNA 5′ terminator stem and 3′ terminator stem. With the 5′ terminator stem sequence sequestered, the terminator stem-loop does not form, allowing transcription of a target gene positioned 3′ to the terminator sequence.

An antisense genetic construct includes, from 5′ to 3′: a promoter sequence and a sequence encoding an antisense activating RNA. In some embodiments, the antisense genetic construct includes a transcriptional termination sequence after the antisense activating RNA sequence. In some embodiments, the antisense activating RNA is at least 5, at least 6, at least 7, at least 8, at least 9, or at least 10 nucleotides in length, and not more than 50, 100, 150, 200, 250, or 300 nucleotides in length. Thus the antisense activating RNA can have a length in the range of 5 to 300 nucleotides, 6 to 200 nucleotides, 7 to 150 nucleotides, 8 to 100 nucleotides, or 10 to 50 nucleotides. In other embodiments, the antisense sRNA molecule is about 97, 96, 92, 91, 90, 88, 82, 80, 78, 72, 71, 70, 68, 64, 62, 58, 53, 52, 50, 48, 45, 40, 38, 37, 36, 35, 33, 32, 29, 28, 26, or 17 nucleotides in length. Specific examples of antisense genetic construct sequences are listed in Table 3.

The antisense activating RNA preferably is a highly linear molecule, that is, the antisense activating RNA has minimal secondary structure resulting from self-annealing portions. These linear RNA sequences are characterized by having a low content of complementary sequences, such as less than 20%, less than 15%, less than 10%, less than 5%, or less than 1% complementary sequences of more than four nucleotides at positions that could contact one another within the RNA strand, thus avoiding formation of secondary structures such as hairpin-loops. See, for example, SEQ ID NOS: 110-114.

In some embodiments, the antisense sequence has at least 85%, at least 90%, at least 93%, at least 95%, at least 98%, or 100% identity to a nucleic acid sequence selected from SEQ ID NOS: 42-87 and 110-114, or SEQ ID NOS: 42-83 and 110-114.

The antisense genetic construct has a promoter. The promoter can be constitutive, inducible, or tissue-specific, as described in detail in other sections of this application.

In some embodiments, the promoter for the activating antisense genetic construct is different from the promoter for the sense genetic construct. For example, the sense genetic construct may have a constitutive promoter, while the antisense genetic construct can have an inducible promoter. In another example, the sense and antisense genetic constructs can each be driven by a constitutive promoter, where each construct is driven by the same promoter, or each construct is driven by a different constitutive promoter.

### Anti-Anti-Terminating STARs

In another example, the sense genetic construct includes, from 5′ to 3′: a promoter, an anti-anti-terminator sequence, an interaction sequence, an anti-terminator sequence encoding an RNA that is substantially complementary to the RNA sequence of the anti-anti-terminator and is also substantially complementary to the 5′ terminator stem of the terminator RNA sequence; a terminator sequence comprising a 5′ terminator stem sequence and a 3′ terminator stem sequence that are substantially complementary to each other, and sequence encoding a poly-uracil RNA sequence. The interaction sequence is an sRNA recognition sequence that facilitates binding of the antisense activating RNA to the terminator sequence. In the absence of the antisense molecule, the anti-anti-terminator sequence and anti-terminator sequence self-pair, and the 5′ terminator stem pairs with the 3′ terminator stem sequence to form a terminator stem-loop DNA secondary structure that prevents.

The antisense activating RNA to an anti-anti-terminator sequence is complementary to the anti-anti-terminator and interaction sequences. When the antisense RNA is transcribed, the antisense RNA binds to/sequesters the anti-anti-terminator and interaction sequences, thus leaving the anti-terminator sequence unpaired. The unpaired anti-terminator sequence then binds to the 5′ terminator sequence. With the 5′ terminator stem paired with/sequestered by the anti-terminator sequence, the terminator stem-loop does not form, and transcription of the target gene is activated. In some embodiments, the sense terminator sequence has at least 85%, at least 90%, at least 93%, at least 95%, at least 98%, or 100% identity to a nucleic acid sequence selected from SEQ ID NOS: 38-41. In some embodiments, the antisense sequence has at least 85%, at least 90%, at least 93%, at least 95%, at least 98%, or 100% identity to a nucleic acid sequence selected from SEQ ID NOS: 84-87.

### Vectors

The sense genetic construct and the antisense genetic construct may each be incorporated into a vector for expression in a host cell. The sense and antisense constructs can be on different vectors, or a single vector. In one embodiment, the sense genetic construct and the antisense genetic construct are on separate vectors. Specific examples of vectors designed according to the disclosed methods are listed in Table 4.

A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like.

Many vectors useful for transferring exogenous genes into target cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, and pET23D.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of a promoter shown effective for expression in eukaryotic cells.

In some embodiments, the vectors described herein can be integrated into the host cell genome with an integrating vector. Integrating vectors typically contain at least one sequence homologous to a host cell chromosome that allows the vector to integrate, and may contain two homologous sequences flanking the expression vector. An integrating vector may be directed to a specific locus in the host cell by selecting the appropriate homologous sequence for inclusion in the vector. The chromosomal sequences included in the vector may occur either as a single segment in the vector, which results in the integration of the entire vector, or as two segments homologous to adjacent segments in the chromosome and flanking the expression vector in the vector, which results in the stable integration of only the expression vector.

The elements that are typically included in expression vectors also include an autonomous site of replication (ARS), a gene encoding antibiotic resistance to permit selection of bacteria that harbor recombinant plasmids, and unique restriction sites in nonessential regions of the plasmid to allow insertion of genetic constructs.

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of interest.

### Additional Composition Elements—RNA Polymerase, Multi-Terminator/Multi-STAR

In some embodiments, the disclosed composition includes an RNA polymerase that will recognize the promoter sequence of the sense and/or antisense genetic constructs and initiate transcription of these genetic constructs. The RNA polymerase may be endogenous to the host or may be introduced by genetic engineering into the host, either as part of the host chromosome or on an episomal element, including a plasmid containing the DNA encoding an RNA polymerase from bacterial or bacteriophage systems, such as a T7, T5, or T3 bacteriophage polymerase, SP6 bacteriophage polymerase, U6 bacteriophage polymerase, H1 human polymerase, or Bst bacterial polymerase. In one example, the polymerase is T7 polymerase.

In some embodiments, the disclosed composition includes one or more sense genetic constructs having at least two terminator sequences in tandem, such as 2, 3, 4, 5, 6, 7, 8, 9, or 10 or more terminators in tandem. Examples of such multi-terminator/multi-sense genetic constructs include two or more distinct sense genetic constructs, each having at least one terminator sequence, or at least one sense genetic construct having at least two terminator sequences in tandem.

This embodiment with multiple terminator/sense genetic constructs can also include at least two antisense constructs, such as 2, 3, 4, 5, 6, 7, 8, 9, or 10 or more antisense constructs. The at least two antisense constructs can include, for example, at least two antisense activating RNAs; at least two genetic constructs encoding at least two activating RNAs; or at least one antisense activating RNA and at least one activating genetic construct encoding an activating RNA. These multi-terminator/multi-activator compositions allow layers of regulation, by combining multiple sense/antisense constructs for additional control of target gene transcription.

Multi sense constructs are able to perform signal integration, whereby a gene of interest is only expressed when all inputs are present (equivalent to Boolean logic gates). STAR sense can also be combined with transcriptional repressors, so that a gene is only expressed when STAR antisense are present and not transcriptional repressor antisense RNAs. Applications for these type of signal integration include biosensing applications, where many input signals can be processed to give a desired output.

### Methods of Regulating Transcription

Further disclosed herein are methods of regulating expression of a gene of interest, by placing the gene of interest under control of the compositions disclosed herein.

In one embodiment, the sense genetic construct is integrated into a genomic sequence upstream of a gene of interest in the host cell. In the absence of the antisense activating RNA, transcription of the gene of interest is repressed, but in the presence of the antisense activating RNA, transcription of the gene of interest is activated. In this embodiment, the antisense activating RNA construct can be an antisense activating RNA that is introduced into the cell, a genetic construct encoding an antisense activating RNA that is also integrated into the genome of the host cell, or a genetic construct encoding an antisense activating RNA that is maintained in the host cell as an extrachromosomal genetic element, such as a plasmid.

In another embodiment, the sense genetic construct is provided on a non-integrating extrachromosomal genetic element, such as a plasmid. In this embodiment, the antisense activating RNA construct can be an antisense activating RNA that is introduced into the cell, or a genetic construct encoding an antisense activating RNA that is also maintained in the host cell as an extrachromosomal genetic element.

Compositions as disclosed herein can be introduced into a host cell to regulate expression of a gene of interest. In some embodiments, the cell is a prokaryotic or bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus.

Alternatively, the composition can be introduced into a eukaryotic cell in vitro, such as a fungal cell, an algal cell, a plant cell, or a mammalian cell. In other embodiments the cell is a fungal cell such as yeast cells, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. Other non-limiting examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In further embodiments that cell is an insect cell, a plant cell, or a mammalian cell, such as a human or mouse cell. In one embodiment, the sense genetic construct is integrated into the genome under transcriptional control of a suitable eukaryotic promoter and/or enhancer elements.

Further disclosed are methods of increasing the transcription of a target gene, by introducing the disclosed compositions into a host cell so that the sense genetic construct is in operable linkage with the target gene and expression of the antisense activating construct increases transcription of the target gene. In one example, a sense genetic construct of the invention is integrated into the genome of a host cell in operable linkage with a target gene, but at least 50%, at least 60%, at least 70%, at least 80%, at least 90%, or 100% of the endogenous regulation of target gene expression is maintained by the endogenous host transcription machinery. In this example, the antisense activating RNA, alone or in combination with a suitable polymerase that recognizes the promoter of the sense genetic construct, increases expression of the target gene, above endogenous levels, by at least 10%, at least 20%, at least 30%, at least 40%, or at least 50% or more.

In one embodiment, the composition is introduced into a bacterial, yeast, plant, or mammalian cell to improve the metabolic engineering of the cell, such as for industrial fermentation, biofuel or recombinant protein production, or for any cell-based process for which improved metabolic pathways are desired. Metabolic engineering typically requires many rounds of optimization to maximize pathway productivity and yield. Often this process focuses on genetic optimizations to fine-tune enzyme expression levels and increase flux through the desired pathway, while minimizing flux through competing pathways. However, this is often problematic because of the vast multi-dimensional expression space that needs to be screened, and the lack of regulators that can cover the necessary range of expression levels.

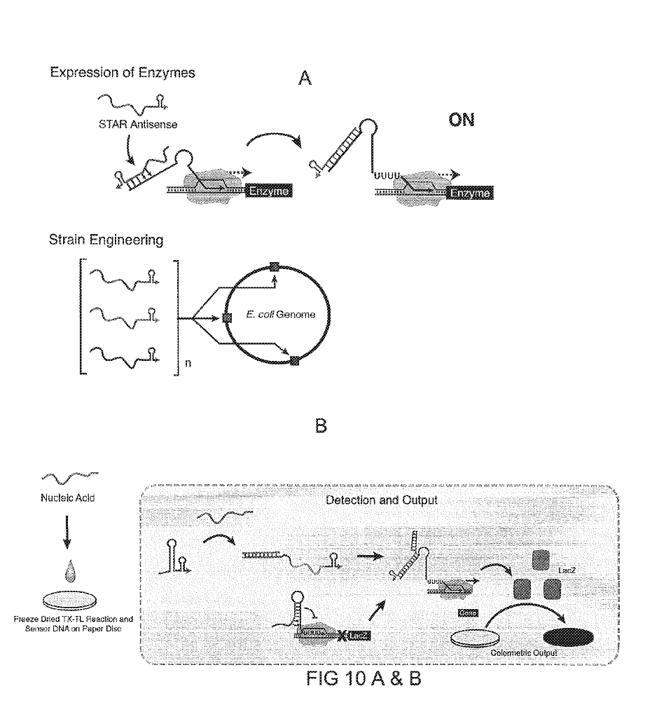

STAR regulation can be utilized to increase flux and yield of desired metabolic pathways. For example, STARs could be used to express an enzyme or enzymes of one or more metabolic pathways. Different STARs can be used combinatorially to express individual enzymes. Different strength STARs can be used to differentially express individual enzymes. STAR antisense/sense can be expressed from inducible promoters to induce expression of individual enzymes. Together these technologies can be used to differentially express enzymes of metabolic pathways, to establish optimal expression regimes (FIG. 10A). STAR antisense can also be used to activate endogenous intrinsic terminators to activate endogenous genes for strain engineering for optimal metabolic pathway performance. Sense RNAs can also be integrated in front of endogenous genes on the chromosome that would be activated by STAR antisense.

STARs Combined with Other RNA Regulatory Constructs Such as RNAi, Riboswitches, and CRISPR

Further disclosed herein are combined systems for RNA-based regulation of gene expression utilizing combinations of STAR activating antisense mechanisms and other RNA-based mechanisms. Examples include STAR sense-antisense regulation combined with one or more of: sRNA, RNAi, or siRNA antisense repression; activation or repression by ligand or co-factor-induced riboswitches; and/or clustered regulatory interspaced short palindromic repeat (CRISPR) regulation. CRISPR interference (CRISPRi) relies upon the use of CRISPR small guide RNAs, in combination with a dead catalytic mutant of the CRISPR Cas9 protein (dCas9), to target specific DNA sequences for transcriptional repression or activation in a variety of organisms. STARs and RNA interference mechanisms such as sRNA, RNAi, siRNA, riboswitches, and CRISPRi, all regulate gene expression via RNA-guided targeting, but are mechanistically distinct. Therefore, these regulatory elements represent complementary components of the RNA synthetic biology toolbox.

In one embodiment, the antisense activating construct targets a riboswitch, or other endogenous RNA regulatory sequence upstream of a target gene, for activation. In other embodiments, the sense genetic construct mimics or replaces an endogenous RNA regulatory sequence, such as a riboswitch, aptazyme, or a sequence that can be recognized by endogenous repressive antisense molecules such as sRNA or RNAi, and the antisense activating RNA activates expression of the target gene.

In one example, sense RNAs are placed upstream of other regulator RNAs, including, but not limited to, CRISPR small guide RNAs, RNAi, or riboswitches. This enables STAR antisense RNA to be used to activate the expression of these regulatory RNAs, creating layered regulation.

### In Vitro Transcription-Translation

Molecular machinery can be engineered to create biosensors that report on the environment through the expression of measurable reporter genes, such as through in vitro cell-free transcription and translation (TX-TL) reactions that can express genetically encoded biosensors. TX-TL reactions utilize a buffered cell lysate that contains gene expression machinery that can transcribe and translate genes encoded from a supplied DNA template.

STAR antisense can be designed to detect and report on the presense of other nucleic acids, such as other RNAs, whereby in the absence of the nucleic acid to be detected, STAR antisense folds to be inactive, whilst in the presence of the nucleic acid to be detected, the STAR antisense is able to bind, causing structural rearrangements that form its active state. In the active state, the STAR antisense can activate its sense RNA. Sense RNAs can be designed to regulate the expression of detectable reporter genes, such as luciferase enzymes, fluorescent proteins and beta-galactosidase. Examples of nucleic acids to be detected include viral RNA genomes as well as cellular messenger RNAs. These STAR sensors can be utilized, for example, in TX-TL reactions (FIG. 10B).

For a TX-TL assay, the basic components for reaction are: a cell extract; a reporter genetic construct that includes a sense genetic construct in operable linkage to a detectable reporter gene; and an activating antisense construct. Additional components can include one or more of: a suitable buffer or dehydrated buffer components, including for example Mg-glutamate, K-glutamate, and dithiothreitol (DTT); amino acids; ATP; GTP; CTP; UTP; tRNA; CoA; NAD; cAMP; folinic acid; spermidine; PGA; and a polymerase (suitable polymerases are disclosed in this specification and known in the art). For example, cell extract and reaction buffer can be prepared according to Sun, Z. Z. et al., J. Vis. Exp. 16:e50762 (2013). Freeze-dried TX-TL reaction components can be stored on filter paper for up to a year before rehydrating with an aqueous solution to activate expression (Pardee et al., Cell 159:940-954 (2014)). In this way, DNA encoding biosensors and TX-TL machinery can be easily stored and later, activated to report on the presence of analytes through expression of a colorimetric, fluorescent, or other reporter output.

Methods to Design STARs

The inventors have determined that the natural log of the observed gene expression is linearly related to the difference in free energy between the initial state and formation of the sense-antisense RNA “seed” complex (SC), according to Equation 1:

ln(gene expression)˜ΔG_{STAR}+ΔG_{Sense}−ΔG_{SC}.

ΔG_{STAR}=the minimum free energy (MFE) of the full STAR antisense molecule. This energy can be calculated using a calculation program, for example, the RNAStructure Fold algorithm (Reuter, J., et al., BMC Bioinformatics. 11:129 (2010)) and an experimental temperature parameter of 37° C.

ΔG_{Target}=the MFE of the sense RNA sequence up until the loop of the terminator stem. This energy can be calculated for the relevant sequence as for the antisense molecule, such as using the RNAStructure Fold algorithm at 37° C.

ΔG_{SC}=The duplex binding energy of the sense-antisense RNA seed complex as discussed below. This energy can be calculated for the relevant sequences using, for example, the RNAStructure DuplexFold algorithm (Reuter, J., et al., BMC Bioinformatics. 11:129 (2010)) at 37° C.

This model can be used to analyze the sequence-function relationships of STAR performance and to design new sense-activating antisense pairs.

### EXAMPLES

### Example 1. Materials and Methods

Plasmid assembly. All plasmids used in this study can be found in Table 1 with key sequences provided in Tables 2, 3 and 4. All sense and antisense plasmids were constructed using inverse PCR (iPCR). All sense plasmids had the p15A origin and chloramphenicol resistance, and all antisense plasmids had the ColE1 origin and ampicillin resistance. All assembled plasmids were verified using DNA sequencing. Superfolder green fluorescent protein (SFGFP) is described in Pédelacq, J. D., et al., Nat. Biotechnol. 24, 79-88 (2006).

TABLE 1
Plasmids. Constitutive promoter BBa_J23119 was obtained from the iGEM
Registry of Standard Biological Parts.

| Plasmid # | Plasmid architecture | Name |
|---|---|---|
| JBL2024 | J23119 - T181.S1 - SFGFP - TrrnB - CmR - p15A origin | T181.S1 |
| JBL2020 | J23119 - T181.S2 - SFGFP - TrrnB - CmR - p15A origin | T181.S2 |
| JBL2022 | J23119 - T181.S3 - SFGFP - TrrnB - CmR - p15A origin | T181.S3 |
| JBL2030 | J23119 - T181.S4 - SFGFP - TrrnB - CmR - p15A origin | T181.S4 |
| JBL2071 | J23119 - T181.S5 - SFGFP - TrrnB - CmR - p15A origin | T181.S5 |
| JBL2147 | J23119 - T181.S6 - SFGFP - TrrnB - CmR - p15A origin | T181.S6 |
| JBL2148 | J23119 - T181.S7 - SFGFP - TrrnB - CmR - p15A origin | T181.S7 |
| JBL2149 | J23119 - T181.S8 - SFGFP - TrrnB - CmR - p15A origin | T181.S8 |
| JBL2150 | J23119 - T181.S9 - SFGFP - TrrnB - CmR - p15A origin | T181.S9 |
| JBL2151 | J23119 - T181.S10 - SFGFP - TrrnB - CmR - p15A origin | T181.S10 |
| JBL2152 | J23119 - T181.S11 - SFGFP - TrrnB - CmR - p15A origin | T181.S11 |
| JBL2021 | J23119 - T181.A1 - t500 - ColE1 origin - AmpR | T181.A1 |
| JBL2037 | J23119 - T181.A2 - t500 - ColE1 origin - AmpR | T181.A2 |
| JBL2064 | J23119 - T181.A3 - t500 - ColE1 origin - AmpR | T181.A3 |
| JBL2128 | J23119 - T181.A4 - t500 - ColE1 origin - AmpR | T181.A4 |
| JBL2153 | J23119 - T181.A5 - t500 - ColE1 origin - AmpR | T181.A5 |
| JBL2154 | J23119 - T181.A6 - t500 - ColE1 origin - AmpR | T181.A6 |
| JBL2155 | J23119 - T181.A7 - t500 - ColE1 origin - AmpR | T181.A7 |
| JBL2156 | J23119 - T181.A8 - t500 - ColE1 origin - AmpR | T181.A8 |
| JBL2157 | J23119 - T181.A9 - t500 - ColE1 origin - AmpR | T181.A9 |
| JBL2158 | J23119 - T181.A10 - t500 - ColE1 origin - AmpR | T181.A10 |
| JBL2109 | J23119 - AD1.S1 - SFGFP - TrrnB - CmR - p15A origin | AD1.S1 |
| JBL2110 | J23119 - IP501.S1 - SFGFP - TrrnB - CmR - p15A origin | IP501.S1 |
| JBL2111 | J23119 - CF10.S1 - SFGFP - TrrnB - CmR - p15A origin | CF10.S1 |
| JBL2198 | J23119 - AD1.S2 - SFGFP - TrrnB - CmR - p15A origin | AD1.S2 |
| JBL2199 | J23119 - AD1.S3 - SFGFP - TrrnB - CmR - p15A origin | AD1.S3 |
| JBL2200 | J23119 - AD1.S4 - SFGFP - TrrnB - CmR - p15A origin | AD1.S4 |
| JBL2801 | J23119 - AD1.S5 - SFGFP - TrrnB - CmR - p15A origin | AD1.S5 |
| JBL2802 | J23119 - AD1.S6 - SFGFP - TrrnB - CmR - p15A origin | AD1.S6 |
| JBL2803 | J23119 - AD1.S7 - SFGFP - TrrnB - CmR - p15A origin | AD1.S7 |
| JBL2115 | J23119 - AD1.A1 - t500 - ColE1 origin - AmpR | AD1.A1 |
| JBL2116 | J23119 - IP501.A1 - t500 - ColE1 origin - AmpR | IP501.A1 |
| JBL2117 | J23119 - CF10.A1 - t500 - ColE1 origin - AmpR | CF10.A1 |
| JBL2804 | J23119 - AD1.A2 - t500 - ColE1 origin - AmpR | AD1.A2 |
| JBL2805 | J23119 - AD1.A3 - t500 - ColE1 origin - AmpR | AD1.A3 |
| JBL2806 | J23119 - AD1.A4 - t500 - ColE1 origin - AmpR | AD1.A4 |
| JBL2807 | J23119 - AD1.A5 - t500 - ColE1 origin - AmpR | AD1.A5 |
| JBL2808 | J23119 - AD1.A6 - t500 - ColE1 origin - AmpR | AD1.A6 |
| JBL2809 | J23119 - AD1.A7 - t500 - ColE1 origin - AmpR | AD1.A7 |
| JBL2183 | J23119 - metH.S1 - SFGFP - TrrnB - CmR - p15A origin | metH.S1 |
| JBL2185 | J23119 - xpt.S1 - SFGFP - TrrnB - CmR - p15A origin | xpt.S1 |
| JBL2184 | J23119 - pbuE.S1 - SFGFP - TrrnB - CmR - p15A origin | pbuE.S1 |
| JBL2819 | J23119 - pbuE.S2 - SFGFP - TrrnB - CmR - p15A origin | pbuE.S2 |
| JBL2818 | J23119 - pbuE.S3 - SFGFP - TrrnB - CmR - p15A origin | pbuE.S3 |
| JBL2817 | J23119 - pbuE.S4 - SFGFP - TrrnB - CmR - p15A origin | pbuE.S4 |
| JBL2816 | J23119 - pbuE.S5 - SFGFP - TrrnB - CmR - p15A origin | pbuE.S5 |
| JBL2815 | J23119 - pbuE.S6 - SFGFP - TrrnB - CmR - p15A origin | pbuE.S6 |
| JBL2190 | J23119 - metH.A1 - t500 - ColE1 origin - AmpR | metH.A1 |
| JBL2192 | J23119 - xpt.A1 - t500 - ColE1 origin - AmpR | xpt.A1 |
| JBL2191 | J23119 - pbuE.A1 - t500 - ColE1 origin - AmpR | pbuE.A1 |
| JBL2824 | J23119 - pbuE.A2 - t500 - ColE1 origin - AmpR | pbuE.A2 |
| JBL2823 | J23119 - pbuE.A3 - t500 - ColE1 origin - AmpR | pbuE.A3 |
| JBL2822 | J23119 - pbuE.A4 - t500 - ColE1 origin - AmpR | pbuE.A4 |
| JBL2821 | J23119 - pbuE.A5 - t500 - ColE1 origin - AmpR | pbuE.A5 |
| JBL2820 | J23119 - pbuE.A6 - t500 - ColE1 origin - AmpR | pbuE.A6 |
| JBL2828 | J23119 - cstA.S1 - SFGFP - TrrnB - CmR - p15A origin | cstA.S1 |
| JBL2981 | J23119 - cstA.A1 - t500 - ColE1 origin - AmpR | cstA.A1 |
| JBL2842 | J23119 - gpt.S1 - SFGFP - TrrnB - CmR - p15A origin | gpt.S1 |
| JBL2986 | J23119 - gpt.A1 - t500 - ColE1 origin - AmpR | gpt.A1 |
| JBL2843 | J23119 - rmf.S1 - SFGFP - TrrnB - CmR - p15A origin | rmf.S1 |
| JBL2988 | J23119 - rmf.A1 - t500 - ColE1 origin - AmpR | rmf.A1 |
| JBL2844 | J23119 - ribA.S1 - SFGFP - TrrnB - CmR - p15A origin | ribA.S1 |
| JBL2983 | J23119 - ribA.A1 - t500 - ColE1 origin - AmpR | ribA.A1 |
| JBL3405 | J23119 - ribA.A2 - t500 - ColE1 origin - AmpR | ribA.A2 |
| JBL3401 | J23119 - ribA.A3 - t500 - ColE1 origin - AmpR | ribA.A3 |
| JBL3402 | J23119 - ribA.A4 - t500 - ColE1 origin - AmpR | ribA.A4 |
| JBL2990 | J23119 - ribA.A5 - t500 - ColE1 origin - AmpR | ribA.A5 |
| JBL3403 | J23119 - ribA.A6 - t500 - ColE1 origin - AmpR | ribA.A6 |
| JBL2991 | J23119 - ribA.A7 - t500 - ColE1 origin - AmpR | ribA.A7 |
| JBL3404 | J23119 - ribA.A8 - t500 - ColE1 origin - AmpR | ribA.A8 |
| JBL007 | J23119 - pT181.H1 - SFGFP - TrrnB - CmR - p15A origin | pT181.H1 |
| JBL1020 | J23119 - Fusion 6 - SFGFP - TrrnB - CmR - p15A origin | Fusion 6 |
| JBL1080 | J23119 - Fusion 4m1 - SFGFP - TrrnB - CmR - p15A origin | Fusion 4m1 |
| JBL1126 | J23119 - Fusion 4 - SFGFP - TrrnB - CmR - p15A origin | Fusion 4 |
| JBL008 | J23119 - pT181.H1 - t500 - ColE1 origin - AmpR | pT181.H1 |
| JBL1029 | J23119 - Fusion 6 - t500 - ColE1 origin - AmpR | Fusion 6 |
| JBL1081 | J23119 - Fusion 4m1 - t500 - ColE1 origin - AmpR | Fusion 4m1 |
| JBL1033 | J23119 - Fusion 4 - t500 - ColE1 origin - AmpR | Fusion 4 |
| JBL2952 | J23119 - Anti.anti.S4 - AD1.A5 - SFGFP - TrrnB - CmR - | A AND B gate |
| | p15A origin | |
| JBL2953 | J23119 - Anti.anti.A4 - AD1.A5 - t500 - ColE1 origin - AmpR | (A, B) |
| | | antisense |
| JBL2901 | J23119 - AD1.S5 - pT181.H1 - SFGFP - TrrnB - CmR - p15A | A AND NOT B |
| | origin | gate |
| JBL2139 | J23119 - pT181.H1 - AD1.A5 - t500 - ColE1 origin - AmpR | (A, B) |
| | | antisense |
| JBL001 | TrrnB - CmR - p15A origin | No - sense |
| | | control |
| JBL002 | J23119 - TrrnB - ColE1 origin - AmpR | No - antisense |
| | | control |
| JBL1860 | J23119 - Anti - anti.S1 - SFGFP - TrrnB - CmR - p15A origin | Anti - anti.S1 |
| JBL1862 | J23119 - Anti - anti.S2 - SFGFP - TrrnB - CmR - p15A origin | Anti - anti.S2 |
| JBL1841 | J23119 - Anti - anti.S3 - SFGFP - TrrnB - CmR - p15A origin | Anti - anti.S3 |
| JBL1864 | J23119 - Anti - anti.S4 - SFGFP - TrrnB - CmR - p15A origin | Anti - anti.S4 |
| JBL1861 | J23119 - Anti - anti.A1 - TrrnB - ColE1 origin - AmpR | Anti - anti.A1 |
| JBL1863 | J23119 - Anti - anti.A2 - TrrnB - ColE1 origin - AmpR | Anti - anti.A2 |
| JBL1842 | J23119 - Anti - anti.A3 - t500 - ColE1 origin - AmpR | Anti - anti.A3 |
| JBL1865 | J23119 - Anti - anti.A4 - t500 - ColE1 origin - AmpR | Anti - anti.A4 |

Abbreviations:
CmR = chloramphenicol resistance cassettes,
AmpR = Ampicilin resistance cassettes,
SFGFP = SuperFolder GFP,
TrrnB = rrnB (E. coli 16S ribosomal RNA B operon) transcription terminator,
T500 = E. coli T500 RNA polymerase transcription terminator.

Strains, Growth Media and In Vivo Bulk Fluorescence Measurements.

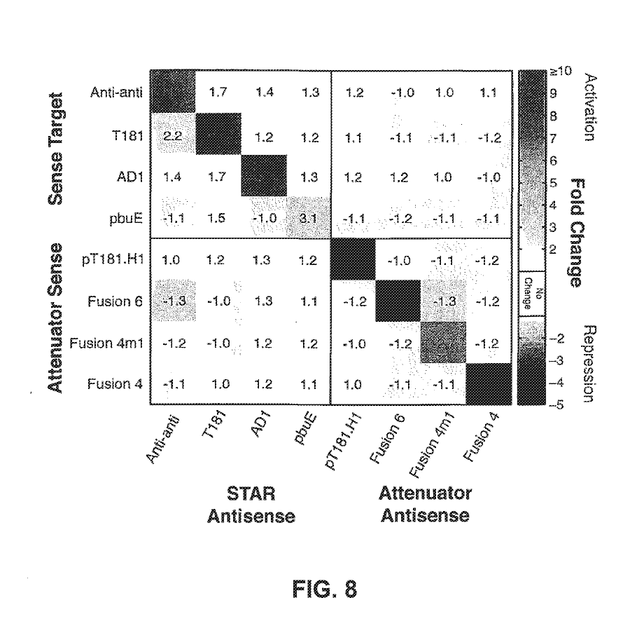

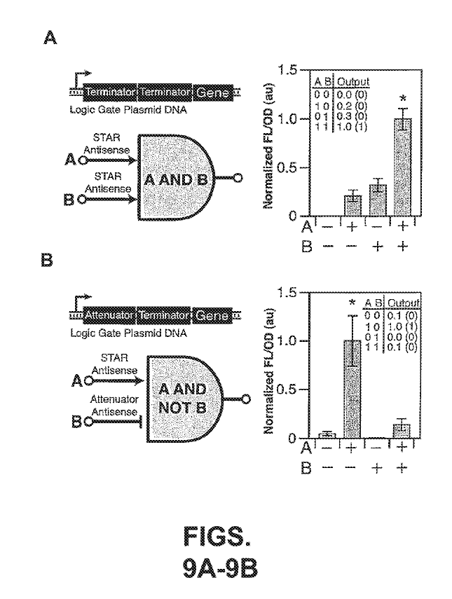

Fluorescence measurement experiments were performed in E. coli strain TG1. Experiments were performed for nine biological replicas collected over three separate days unless otherwise stated in the figure legend. For each day of in vivo bulk fluorescence measurements, plasmid combinations were transformed into chemically competent E. coli TG1 cells and plated on LB+Agar (Difco) plates containing 100 mg/ml carbenicillin and 34 mg/ml chloramphenicol and incubated approximately 17 h overnight at 37° C. Plates were taken out of the incubator and left at room temperature for approximately 7 h. Three colonies were used to inoculate three cultures of 300 μl of LB containing carbenicillin and chloramphenicol at the concentrations indicated above in a 2-ml 96-well block (Costar), and they were grown for approximately 17 h overnight at 37° C. at 1,000 r.p.m. in a VorTemp 56 (Labnet) bench top shaker. Four microliters of each overnight culture were then added to 196 μl (1:50 dilution) of supplemented M9 minimal medium (1×M9 minimal salts, 1 mM thiamine hydrochloride, 0.4% glycerol, 0.2% casamino acids, 2 mM MgSO₄, 0.1 mM CaCl₂) containing the selective antibiotics. Cells were then grown for 4 h for all data except for those in FIGS. 8 and 9, for which cells were grown for 5 h in the same conditions as the overnight culture. Fifty microliters of this culture were then transferred to a 96-well plate (Costar) containing 50 μl of phosphate-buffered saline (PBS). SFGFP fluorescence (FL; 485 nm excitation, 520 nm emission) and optical density (OD) at 600 nm were then measured using a SynergyH1 plate reader (Biotek).

Bulk Fluorescence Data Analysis.

On each 96-well block, there were two sets of controls: a medium blank (M9) and E. coli TG1 cells that do not produce SFGFP (transformed with control plasmids JBL001 and JBL002). The block contained three replicates of each control. OD and FL values for each colony were first corrected by subtracting the corresponding values of the medium blank. The ratio of FL to OD (FL/OD) was then calculated for each well (grown from a single colony), and the mean FL/OD of TG1 cells without SFGFP was subtracted from each colony's FL/OD value. Three biological replicas were collected from one independent transformation, with three colonies characterized per transformation (nine colonies total). Mean FL/OD values were calculated over replicas, and error bars represent s.d. For characterization of orthogonality (FIG. 8), the fold change (activation or repression) for each pair was determined by dividing the FL/OD of cells containing both the sense and antisense plasmids (ON) by the FL/OD of cells containing the sense plasmid and a no-antisense control plasmid (OFF). If this number was less than 1, indicating repression, the negative reciprocal was taken to give the fold repression, i.e., 0.20 becomes −5-fold repression. A Welch's t-test was performed to determine statistical significance (P<0.05) between different conditions; exact comparisons used are in figure legends.

Total RNA Extraction for Quantitative PCR.

For all extraction of total RNA for quantitative PCR (qPCR) experiments, E. coli strain TG1 was used. Plasmids were transformed, and subsequent colonies were grown overnight as described for in vivo bulk fluorescence measurements. For each biological replica, 20 μl of a single overnight culture was added to three wells containing 980 μl (1:50 dilution) of supplemented M9 minimal medium containing the selective antibiotics and grown for 4 h at the same conditions as the overnight cultures. For each plasmid combination, 500 μl of cells were removed from three wells (grown from one colony) and combined into a 1.6-ml tube and pelleted by centrifugation at 13,000 r.p.m. for 1 min. The supernatant was removed, and the remaining pellet was resuspended in 750 μl of Trizol reagent (Life Technologies), homogenized by repetitive pipetting, incubated at room temperature for 5 min and stored at −80° C. for approximately 17 h. These samples were defrosted on ice, 150 μl of chloroform (Sigma Aldrich) was added, and the samples were mixed for 15 s and incubated at room temperature for 3 min. Following incubation, the samples were centrifuged for 15 min at 12,000 g at 4° C., and 200 μl of the top aqueous layer was removed. One microliter of glycogen (20 μg/μl; Life Technologies) and 375 μl of isopropanol were added to the aqueous phase, and the sample was incubated at room temperature for 10 min and centrifuged for 15 min at 15,000 r.p.m. at 4° C. Following centrifugation, the isopropanol was carefully removed from the total RNA/glycogen pellets, washed in 600 μl of chilled 70% ethanol (EtOH) and centrifuged for 2 min at 15,000 r.p.m. at 4° C. EtOH was removed, and tubes were centrifuged for another 2 min at 15,000 r.p.m. at 4° C. to ensure that all of the ethanol was effectively removed. Pellets were resuspended in 20 μl of RNase free double-distilled water (ddH₂0).

DNase Treatment of Total RNA for qPCR.

Purified total RNA samples were quantified by the Qubit Fluorometer (Life Technologies) and were diluted to a concentration of 10 ng/μl in a total of 10 μl RNase free ddH₂0 and digested by Turbo DNase (Life Technologies) according to the manufacturer's protocol. After digestion, 150 μl of RNase free ddH₂0 and 200 μl phenol/chloroform (Acros Organics) was added, and the sample was vortexed for 10 s and incubated for 3 min at room temperature and centrifuged for 10 min at 15,000 r.p.m. at 4° C. After centrifugation, 190 μl of the top aqueous layer was carefully removed, 190 μl of chloroform was added, and samples were vortexed for 10 s, incubated for 3 min at room temperature and centrifuged for 10 min at 15,000 r.p.m. at 4° C. After centrifugation, 170 μl of the top aqueous layer was carefully removed, 170 μl of chloroform was added, and samples were vortexed for 10 s, incubated for 3 min at room temperature and centrifuged for 10 min at 15,000 r.p.m. at 4° C. After centrifugation, 120 μl of the top aqueous layer was carefully removed and added to 1 μl glycogen, 360 μl of chilled 100% EtOH and 12 μl of 3 M sodium acetate, pH 5. Samples were vortexed for 10 s and stored at −80° C. for 1 h. Samples were then centrifuged for 30 min at 15,000 r.p.m. at 4° C. Supernatant was removed, and the pellets were washed in 600 μl of chilled 70% EtOH. Samples were then centrifuged for 2 min at 15,000 r.p.m. at 4° C., and the EtOH was removed. Samples were recentrifuged for 2 min at 15,000 r.p.m. at 4° C., and residual EtOH was removed, and pellets were air-dried for 10 min and eluted in 10 μl RNase fee ddH₂O.

Normalization of Total RNA, Reverse Transcription and qPCR Measurements.

To enable comparison between different samples, each DNase treated sample was normalized to contain the same total RNA concentration. Each sample was quantified by Qubit Fluorometer, and the sample was diluted to 0.025 ng/μl of total RNA in 20 μl RNase free ddH₂0. One microliter of this total RNA, 1 μl of 2 μM reverse transcription primer, 1 μl of 10 mM of dNTPs (New England Biolabs) and RNase-free ddH₂0 (up to 6.5 μl) were incubated for 5 min at 65° C. and cooled on ice for 5 min. 0.25 μl of Superscript III reverse transcriptase (Life technologies), 1 μl of 100 mM Dithiothreitol (DTT), 1× first-strand buffer (Life technologies), 0.5 μl RNaseOUT (Life Technologies) and RNase free H₂O up to 3.5 μl were then added, and the solution was incubated at 55° C. for 1 h, 75° C. for 15 min and then stored at −20° C. qPCR was performed using 5 μl of Maxima SYBR green qPCR master mix (Thermo Scientific), 1 μl of cDNA and 0.5 μl of 2 μM SFGFP qPCR primers (Table 5) and RNase-free ddH₂O up to 10 μl. A ViiA 7 real-time PCR machine (Applied Biosystems) was used for data collection using the following PCR program: 50° C. for 2 min, 95° C. for 10 min, followed by 30 cycles of 95° C. for 15 s and 60° C. for 1 min. All of the measurements were followed by melting curve analysis. A MicroAmp EnduraPlate Optical 384-well plate (Applied Biosystems) and an Optically Clear seal (Applied Biosystems) were used for all measurements. Results were analyzed using ViiA 7 software (Applied Biosystems) by a relative standard curve. For quantification, a four-point standard curve covering a 1,000-fold range of SFGFP cDNA concentrations was run in parallel and used to determine the relative SFGFP cDNA abundance in each sample. It was shown that the SFGFP qPCR primer set had a primer efficiency between 90-103%. All of the cDNA samples were measured in triplicate, and nontemplate controls run in parallel to control for contamination and nonspecific amplification or primer dimers. In addition, qPCR was performed on total RNA samples to confirm that no DNA plasmid was detected under conditions used. Melting curve analysis was performed to confirm that only a single product was amplified.

Total RNA Extraction for RNA-Seq.

For all extractions of total RNA for RNA-seq experiments, E. coli strain K12 MG1655 was used. Antisense or no-antisense control plasmids were transformed, and subsequent colonies were grown overnight as described for in vivo bulk fluorescence measurements except that liquid and solid media contained only 100 mg/ml carbenicillin. For each biological replica, 20 μl of a single overnight culture was added to three wells containing 980 μl (1:50 dilution) of supplemented M9 minimal medium containing 100 mg/ml carbenicillin and grown for 4 h at the same conditions as the overnight cultures. For each plasmid combination, 2-3 ml of cells were removed from three wells (grown from one colony) and two volumes of RNAprotect bacterial reagent (Qiagen) were added and incubated for 5 min at room temperature. Total RNA was then purified using an RNeasy Mini Kit (Qiagen) according to the manufacturer's protocol and eluted in 50 μl of RNase free ddH₂0 and stored at −80° C.

DNase Treatment of Total RNA for RNA-Seq.

Purified total RNA samples were quantified by Qubit Fluorometer, and 6 μg of total RNA was digested by Turbo DNase according to the manufacturer's protocol and purified as described for DNase treatment of Total RNA for qPCR. The quality of the DNase treated total RNA samples were assessed using a Fragment Analyzer (Advanced Analytical).

rRNA Depletion of Total RNA for RNA Seq.

DNase treated total RNA samples were quantified by Qubit Fluorometer, and 3 μg of RNA from each sample was treated with the Ribo-Zero rRNA removal kit (Gram-negative) (Epicentre) to remove rRNA according to the manufacturer's protocol and eluted in 10 μl RNase free ddH₂O. For each sample, the rRNA removal was assessed using a Fragment Analyzer.

RNA-Seq Library Preparation, Sequencing and Analysis.

rRNA-depleted total RNA samples were quantified by Qubit Fluorometer, and 50 ng of RNA was used to prepare RNA-seq libraries using the ScriptSeq v2 RNA-seq Library Preparation Kit (Epicentre) according to the manufacturer's protocol. The quality of the RNA-seq libraries was accessed using a Fragment Analyzer. Samples were sequenced on a MiSeq (Illumina) following the manufacturer's standard cluster generation and sequencing protocols for 50-bp paired-end reads of sequencing. RNA-seq data sets were analyzed using the Tophat/Cufflinks pipeline using Bowtie version 1.1.0, Tophat version 2.0.12 and Cufflinks version 2.2.1 (Trapnell, C. et al., Nat. Protoc. 7, 562-578 (2012)). To align against the annotated E. coli K-12 MG1655 genome, the ensemble FASTA genomic sequence (.fa) and general feature format (.gff3 file) for the GCA_000005845.2 genome assembly were used. The gff3 annotation file was further manually curated to remove duplicate gene ID entries and then converted to .gtf format using the gffread utility provided in the Cufflinks package. Each set of paired-end sequencing reads for each replicate experiment was aligned to the E. coli K-12 MG1655 genome using tophat options—-no-novel-juncs and—-library-type fr-secondstrand. Differential gene expression was analyzed using cuffdiff with the −u option that specified the same input .gtf file as used in the tophat mapping. Scatter plots were made using CummeRbund version 2.6.1 (Goff, L., et al. (R package version 2.6.1, 2012)).

Calculation of Free Energies for STAR Design Principles.

All AG terms were calculated using the command-line version of RNAStructure v5.5 (Reuter, J., et al., BMC Bioinformatics. 11:129 (2010)). The Fold utility was used to calculate ΔG_{STAR} and ΔG_{Target}, and the DuplexFold utility was used to calculate ΔG_{SC}, both using the default options.

Characterization of STARs in TX-TL.

Cell extract and reaction buffer were prepared according to Sun, Z. Z. et al., J. Vis. Exp. 2013 16, e50762 (2013). Gene expression was optimized via the addition of 5 mM Mg-glutamate and 80 mM K-glutamate. TX-TL buffer and extract tubes were thawed on ice for approximately 20 min. Separate reaction tubes were prepared with combinations of DNA representing a given test condition. Appropriate volumes of DNA, buffer and extract were calculated according to Sun et al., supra. A final concentration of 1 nM sense target plasmid DNA and 10 nM STAR antisense plasmid DNA or 10 nM no-antisense control plasmid DNA was run in triplicate. Buffer and extract were mixed together, incubated at 37° C. for 20 min and then added to each tube of DNA. 10 μl of each TX-TL reaction mixture was transferred to a 384-well plate (Nunc), covered with a plate seal (Nunc) and placed on a SynergyH1 plate reader. The temperature was controlled at 37° C., and SFGFP fluorescence was measured (485 nm excitation, 520 emission) every 5 min.

### Example 2. Engineering STARs Through Direct Anti-Termination

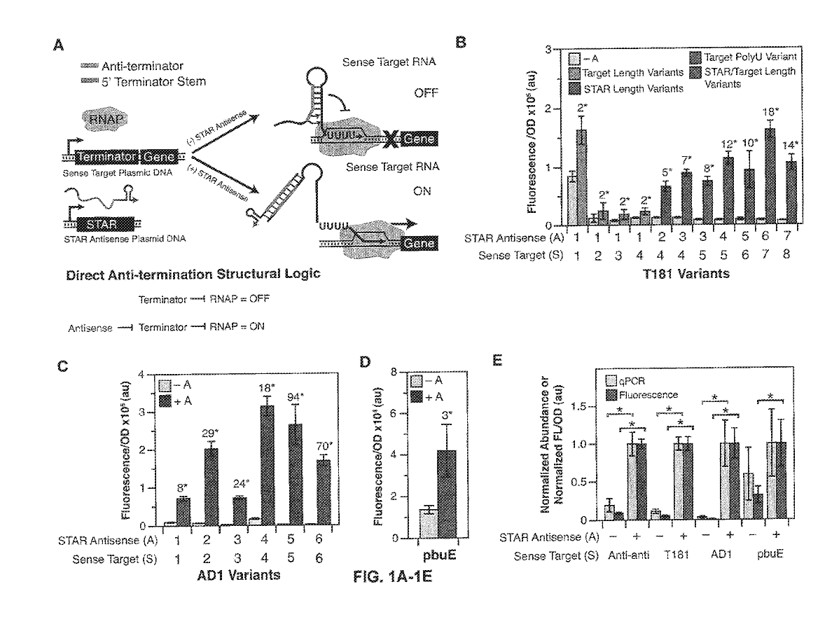

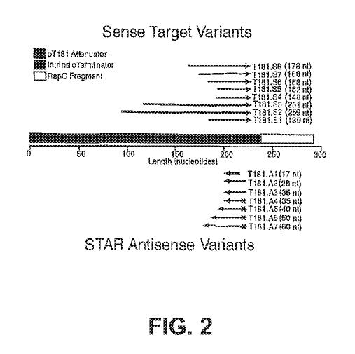

The inventors sought to design short transcription activating RNAs (STARs) that directly act as anti-terminators. In this approach, STARs that contain an anti-terminator sequence are designed to prevent the formation of terminator hairpins placed upstream of coding regions within the target RNA. This has the effect of removing a layer of structural repression, which also accomplished our goal of inverting the overall attenuator function from repression to activation. To implement this design, the inventors began by fusing the sequence encoding the pT181 terminator hairpin upstream of the RBS-SFGFP region (FIG. 1A). In this configuration, the transcription terminator should form by default, preventing downstream transcription of the target RNA. STAR antisenses were designed to contain sequences complementary to the 5′ side of the terminator hairpin, so that when present, they would bind the nascent 5′ terminator region as trans-acting anti-terminators that allow transcription elongation. The inventors initially created a series of STAR antisense and sense target variants that varied in length and sequence composition to achieve up to 12.4-fold (±2.0) activation (T181 A4/S5; FIG. 2).

The inventors added additional complementary RNA sequences to both the STAR antisense and sense targets present upstream of the natural pT181 terminator to increase the potential interaction region between STAR and target. By adding this sequence in ˜10-nt increments, the inventors created six new STAR-target pairs, with T181 A6/S7 showing the strongest activation (18-fold (±3.2); FIG. 1B). Notably, increasing the length of the interaction sequence between the STAR antisense and sense target only improved activation up to a point, which the inventors hypothesized was because of a trade-off between increasing the binding strength of the intermolecular STAR antisense-sense target interaction and increasing the potential interference of intramolecular secondary structures of the individual strands.

### Example 3. Designing STARs for Other Transcriptional Attenuators and Riboswitches

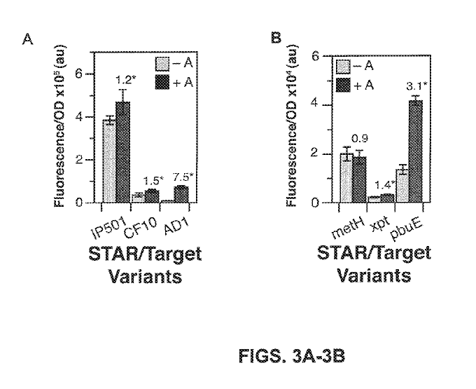

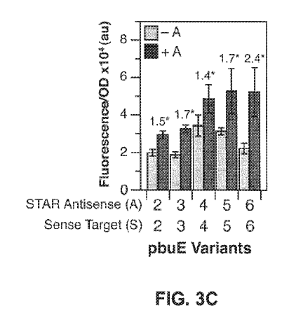

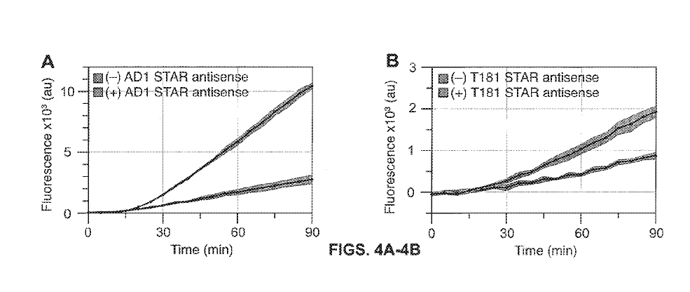

To test whether this approach could be generalized to create additional STAR regulators, the inventors applied this strategy to create STARs that target terminator hairpins from other transcriptional attenuators and transcriptional riboswitches. For transcriptional attenuator mechanisms, the inventors focused on targeting the terminators from the pIP501, pCF10 and pAD1 plasmid attenuation systems. Of these systems, the AD1 A1/S1 pair was the most promising, showing 7.5-fold (±0.8) activation (FIG. 3A). To further optimize this system, the inventors lengthened the STAR antisense-sense target interaction region by adding an additional sequence upstream of the natural terminator to this pair in ˜10-nt increments, as before. In this way, the inventors were able to find a pair (AD1 A5/S5) that displayed 94-fold (±26) activation (FIG. 1C).

For conversion of transcriptional riboswitches into STAR-target pairs, the inventors focused on creating STARs from the terminator hairpins of three well-characterized riboswitches shown to have a high degree of modularity: meth (Ceres, P., et al., Nucleic Acids Res. 41, 10449-10461 (2013)), xpt-pbuX (Ceres, P., et al., ACS Synth. Biol. 2, 463-472 (2013)) and pbuE (Ceres, P., et al., Nucleic Acids Res. 41, 10449-10461 (2013)). Of these, the pbuE STAR showed 3.1-fold (±1.0) activation (FIG. 1D, FIG. 3B). Optimizations were attempted as before, though no greater fold change in activation was achieved by increasing the STAR-target interaction sequence beyond 45 nucleotides (FIG. 3C).

To corroborate that these systems regulate expression through transcriptional activation, the inventors used quantitative PCR (qPCR) to determine the steady-state level of SFGFP mRNA in the presence and absence of STAR antisense expression for the best activators (FIG. 1E). For clarity, the STAR-target RNAs for these systems are denoted anti-anti (Anti-anti.A4/S4), T181 (T181.A6/S7), AD1 (AD1.A5/S5) and pbuE (pbuE.A1/S1). For the anti-anti, T181 and AD1 STAR-target pairs, the inventors observed a statistically significant (P<0.05) increase in the steady-state abundance of SFGFP mRNA in the presence of their STAR antisenses, thus corroborating that these systems operated through transcriptional activation. For these systems, the inventors observed small discrepancies between qPCR quantifications of SFGFP mRNA and the measured SFGFP fluorescence that the inventors attribute to the mass normalization of qPCR samples to total RNA concentrations, which can vary depending on the overall gene expression in each condition tested. The pbuE system showed an overall increase in SFGFP mRNA abundance in the presence of its STAR antisense sequence in the qPCR experiments, though it was not statistically significant (P>0.05). This is most likely because of the low fold activation of this system and the inherent noise of the qPCR experiment.

### Example 4. STAR Activation in In Vitro Transcription/Translation Systems

To further demonstrate that the observed in vivo transcriptional activation of the STAR-target systems is not due to an off-target or nonspecific gene expression response in the cell, the inventors tested their function using in vitro transcription and translation (TX-TL) reactions. TX-TL reactions contain all of the necessary cellular machinery for gene expression but contain no endogenous genomic DNA templates, and so they provide a reduced gene expression system independent of other host genes. Thus STARs are only expected to activate gene expression in TX-TL reactions if their function is not dependent on other genomic targets. The inventors observed activation for the AD1 and T181 direct anti-terminator STARs in TX-TL reactions (FIGS. 4A-4B).

STAR sense/antisense technology can be similarly used as a biosensor in TX-TL diagnostic assays (FIG. 10B). In these assays, the STAR antisense molecule is designed to bind a nucleic acid to be detected and the sense genetic construct is placed upstream of a reporter molecule. The presence of the nucleic acid of interest in a sample alters the conformation of the antisense RNA from an inactive to an active form. The active antisense RNA binds to the sense sequence and activates transcription of the reporter gene, providing a measurable determination of the presence of the nucleic acid to be detected.

### Example 5. Sequence-Function Model of STARs

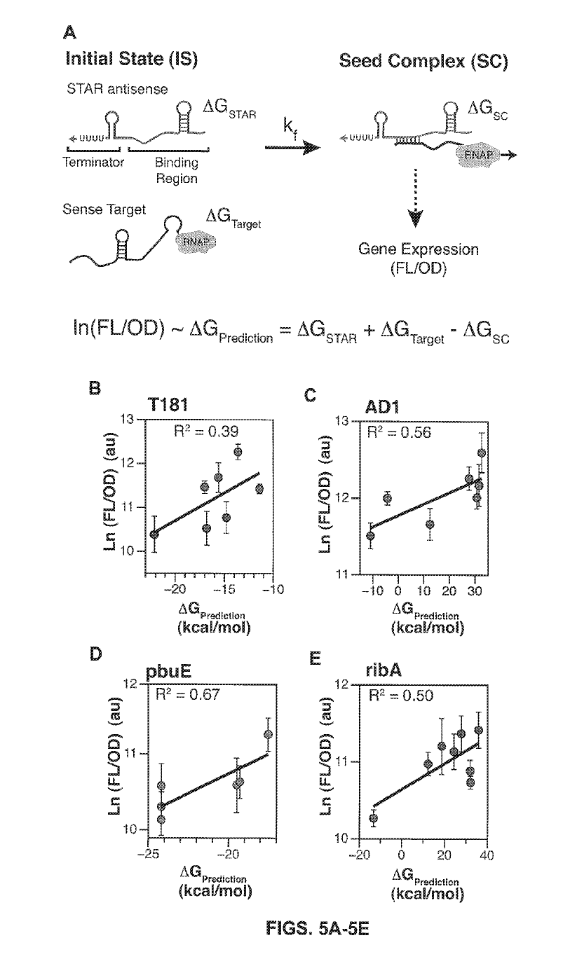

The inventors sought to develop a kinetic model that could explain the range in activation the inventors observed as a function of STAR antisense and target RNA sequence. To develop this model, the inventors first considered the different RNA structural states formed as the STAR antisense interacts with the sense target RNA. The inventors hypothesized the presence of three structural states in the STAR mechanism: the initial state (IS), consisting of the individually folded STAR antisense and sense target; an extended duplex that consists of perfect base pairing between STAR and target; and a seed complex (SC) in which STAR-target interactions are initiated that serves as an intermediate state between the initial state and the extended duplex (FIG. 5A). Because the STAR-mediated transcriptional regulatory decision must happen during transcription elongation by RNAP, the inventors hypothesized that seed complex formation is much faster than extended duplex formation, which has been observed in the pT181 transcriptional repression system. The inventors further hypothesized that seed complex formation is sufficient to prevent the formation of the terminator hairpin and enact the regulatory decision, and thus the rate of overall gene expression is proportional to the rate of seed complex formation.

The inventors sought to establish design rules for direct anti-terminator STAR function by linking the sequences of the STAR antisense and sense target to the observed gene expression in the presence of the STAR antisense (i.e. measured fluorescence). Experimental evidence suggested that the activation of transcription was governed by the STAR antisense binding to the target region before terminator formation. Thus one of the simplest ways to model the STAR mechanism was to consider the bimolecular binding interaction between the STAR antisense and the sense target binding region.

Since the STAR binding region is fully complementary to the target region, theoretically an extended duplex can form between STAR and target. However, evidence from antisense-mediated transcription repression systems suggest that full duplex formation does not form on the short timescales of transcriptional decisions. Instead, the inventors hypothesized the presence of an intermediate “seed complex” (SC) in which the STAR antisense is bound to the target region in a limited seed region that serves to nucleate duplex formation (FIG. 5A). The inventors further hypothesized that the formation of the SC was enough to prevent terminator formation and thus activate transcription. In this case, the rate of transcription of the downstream gene would be directly related to the rate of SC formation.

The inventors predicted that the observed STAR-mediated gene expression was a function of the free energies of the different RNA structural states. Specifically, this analysis predicted that the natural log of the observed gene expression (fluorescence (FL)/optical density (OD)) is linearly related to the difference in free energy between the initial state and the seed complex: ln(FL/OD)˜ΔG_{STAR}+ΔG_{Target}−ΔG_{SC} (equation (1)). This free energy difference naturally reflects the competing effects of intramolecular base pairs within the STAR and target that need to be broken before the formation of intermolecular base pairs that lead to the seed complex and, ultimately, transcription activation.

To test the prediction of equation (1), the inventors calculated AG for each term. The inventors estimated each term as follows:

ΔG_{STAR}=the minimum free energy (MFE) of the full STAR antisense molecule, including the terminator. This energy was calculated with the RNAStructure Fold algorithm (Reuter, J., et al., BMC Bioinformatics. 11:129 (2010)) at 37° C.

ΔG_{Target}=the MFE of the target RNA up until the loop of the terminator stem. This length was chosen under the hypothesis that the STAR binding event happens before full terminator synthesis, so only this portion is available to fold. This energy was calculated for the relevant sequence with the RNAStructure Fold algorithm (((Reuter, J., et al., BMC Bioinformatics. 11:129 (2010)) at 37° C.

ΔG_{SC}=the duplex binding energy of the hypothesized seed region as discussed below. This energy was calculated for the relevant sequences with the RNAStructure DuplexFold algorithm (Reuter, J., et al., BMC Bioinformatics. 11:129 (2010)) at 37° C. Note that this algorithm does not allow intramolecular pairing within the STAR antisense and sense target regions—rather it only calculates the free energy of the RNA-RNA interactions within the seed region.

The inventors then determined which RNA sequences to use in computational folding algorithms to approximate the AG terms, by choosing the length of the sense target strand that comprised the initial state and the region of interaction that characterized the seed complex.

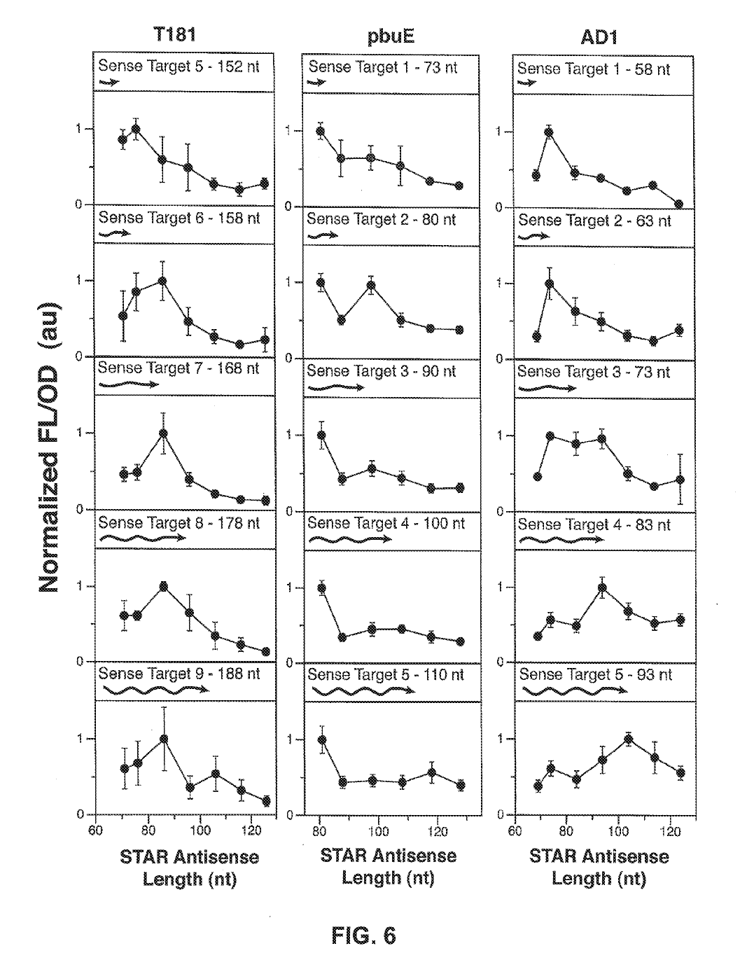

To choose the length of sequence that characterized the seed complex, for each STAR/target system the inventors scanned different length seed complexes in 6-nt increments and compared our predicted correlations between equation (1) and experimental characterization of STAR activator function. For this the inventors used different length STAR antisense variants, and five different length sense targets variants for each of the T181, AD1 and pbuE systems. For each system, the inventors characterized the fluorescence/OD observed from a matrix of STAR and target combinations by challenging each different length sense target with each different length STAR antisense. As predicted by the inventors' model, there was an optimum STAR antisense length above which no further increase in fluorescence was observed for each sense target variant (FIG. 6).

Comparing this fluorescence data to the predictions of eq (1) revealed that to achieve the best correlation between the experimental characterization of fluorescence output and the ΔG_{prediction} (=ΔG_{STAR}+ΔG_{Target} ΔG_{SC}) term, different seed complex lengths were required for STAR activators derived from different systems. For the T181, AD1 and pbuE seed complexes, seed complex lengths of 12-nt, 42-nt and 12-nt gave the best correlation between experimentally observed and predicted function within each comparison. The inventors hypothesized that this observed difference in optimal seed complex length is due to underlying differences in the folding of different sense target RNAs from each system. As the sense target is being actively transcribed when regulatory decisions are made, the inventors believe the co-transcriptional folding of the sense target RNA and the non-uniform dynamics of RNA polymerase (RNAP) transcription are important factors determining the optimum seed complex length.

The inventors observed a consistent positive correlation between the measured STAR-mediated gene expression and the ΔG_{Prediction} term. For the T181, AD1 and pbuE systems used in FIG. 1E, R² was 0.39, 0.56 and 0.67, respectively, and the inventors observed equally strong correlations for predictions when applied to different-length sense target variations of 152, 158, 168, 178, and 188 nt (for T181), 58, 63, 73, 83, and 93 nt (for AD1), and 73, 80, 90, 100, and 110 nt (for pbuE) (FIG. 6). These results indicated that the inventors' kinetic model captured the essence of the STAR direct anti-terminator mechanism.

### Example 6. Designing STARs for Intrinsic Terminators

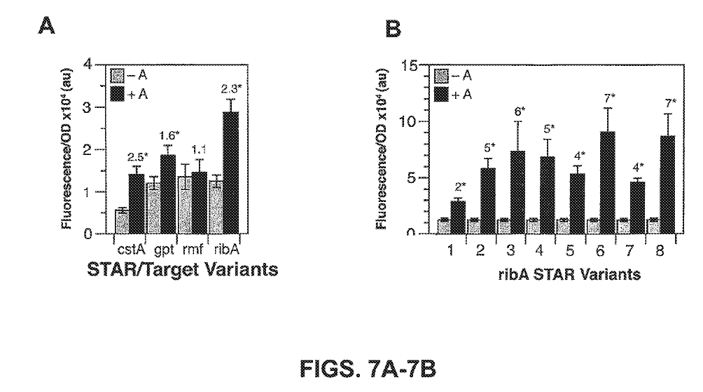

To further validate this model, the inventors sought to use the model to design new STARs that target alternative sources of intrinsic terminators. As initial STAR antisense sequences targeted terminators present in existing transcriptional switches, the inventors chose to focus on targeting intrinsic terminators present in the E. coli genome at the ends of genes to test whether STARs could target this class of terminator. The inventors placed intrinsic terminators upstream of a strong RBS and SFGFP in our two-plasmid system and constructed STAR antisense sequences to target their 5′ halves. Initial screening identified several functional variants, and the strongest activation was a 2.3-fold (±0.37) increase, observed for the intrinsic terminator of the GTP cyclohydrolase II ribA gene (FIG. 7A).

Using their mechanistic model of STAR activation, the inventors designed seven more STAR antisense variants that the inventors predicted would cover a range of activation levels. A comparison of the observed fluorescence caused by these STAR antisense variants with the ΔG_{Prediction} term for this system revealed a good correlation (R²=0.50), consistent with the results from our previous model (FIG. 5E). Furthermore, the largest ΔG_{Prediction} term successfully identified our optimal STAR antisense, which had a 7.2-fold (±1.9) activation of expression (ribA A6, FIG. 7B). It should be noted that because the sense target RNA sequence was constant in this series, the ΔG_{Target} term was removed from our model for these predictions, which amounts to a shift in all ΔG_{Prediction} terms. These results demonstrated that the inventors could successfully apply our model to aid in the design of new STARs and that STARs could be designed to target naturally occurring intrinsic terminators not derived from RNA regulators.

### Example 7. Engineering STARs with an Anti-Anti-Terminator Mechanism

The pT181 attenuator is a sense RNA sequence that regulates transcription elongation through RNA structural rearrangements that either enable or inhibit the formation of an intrinsic transcription terminator hairpin upstream of a coding region. In the absence of an antisense sRNA, the attenuator folds so that an anti-terminator sequence sequesters the 5′ side of the intrinsic terminator hairpin, thereby inhibiting terminator formation and allowing transcription elongation. In its presence, the antisense sRNA interacts with the attenuator region containing the anti-terminator, which enables terminator formation that halts transcription near the beginning of the mRNA.

To inverts the overall attenuator function from repression to activation, an anti-anti-terminator sequence was fused upstream of the attenuator to sequester the anti-terminator itself. To construct such a mechanism that functions in vivo in E. coli cells, the inventors fused designed anti-anti-terminator sequences to the 5′ end of the pT181 attenuator sequence within the sense target RNA. These anti-anti-terminator sequences consisted of a region complementary to the anti-terminator followed by a sRNA recognition sequence taken from modular RNA-RNA interaction domains that the inventors have previously used to construct chimeric transcriptional attenuators. Four variants of the sense target RNA were designed using the anti-anti-terminator mechanism and four different sRNA recognition sequences. For each of these, STAR antisense sequences were designed to bind the sRNA recognition sequence and sequester the anti-anti-terminator so that transcription anti-termination (activation) was achieved.

To characterize transcriptional activation, plasmids were constructed whereby each sense target RNA was placed downstream of a constitutive promoter and upstream of a superfolder GFP (SFGFP) coding sequence with its own RBS. STAR antisense sRNAs were expressed on a separate plasmid from a constitutive promoter and were followed by their own transcription terminators (t500 terminator). A no-antisense control plasmid consisting of the constitutive promoter followed directly by a transcription terminator (rrnB terminator (TrrnB); was also constructed. For each sense target (S) plasmid tested, E. coli cells were transformed together with either the STAR antisense-expressing plasmid (A) or the no-antisense control plasmid, and SFGFP fluorescence (485 nm excitation and 520 nm emission) and optical density (600 nm) were measured for each culture. Of the four designs tested, two showed significant (P<0.05) activation of gene expression in the presence of the STAR antisense, with STAR antisense and sense target pairs 3 and 4 (anti-anti A3/S3 and A4/S4) showing 3.6-fold (±0.3) and 10.8-fold (±1.5) activation, respectively (mean±s.d.). These results demonstrated that the inventors could successfully reengineer the structural logic of a sRNA transcriptional repression mechanism to convert it into a transcriptional activator.

### Example 8. Orthogonality of STARs

The inventors next sought to test whether STARs could be used as components of higher-order RNA regulatory circuitry. A prerequisite to such utility is their orthogonality to each other, i.e., the ability of a STAR antisense to only activate its cognate target without cross-talk to other targets. To determine STAR orthogonality, the inventors measured the fold activation of all possible STAR-target pairs among the best direct anti-terminator activators from the T181, AD1 and pbuE systems and the best anti-anti-terminator mechanism (FIG. 8). Assuming that a onefold change is no activation, the inventors observed a high degree of orthogonality between these activators. The one exception was for the pbuE sense target, which was activated 1.5-fold by the T181 STAR antisense (compared to its cognate, which had 3.1-fold activation), although this result was most likely biased by the overall low fold activation of this activator. The inventors surprisingly observed orthogonality between the two pT181-derived activators, the T181 (direct anti-terminator mechanism) and anti-anti (anti-anti-terminator mechanism) activators, suggesting that the changes to the STAR antisense and sense target pairs were substantial enough to allow independent regulation. The inventors designed and tested an 8×8 orthogonality matrix of attenuator and activator sense and antisense sRNAs, and fold change relative to the no-antisense control was determined (FIG. 8). The inventors observed a high level of orthogonality between the noncognate pairs of sense and antisense from the attenuator and activator systems. Although the inventors observed some small levels of cross-talk, most fold changes were within error of the no-antisense control. These results demonstrate that the STARs are highly orthogonal to themselves and to the existing sRNA transcriptional attenuator libraries, suggesting that these activators in fact expand the versatility of the RNA transcriptional regulatory toolbox required for engineering RNA-only genetic networks.

Another type of orthogonality that is only beginning to be studied in synthetic biology is orthogonality to the host cell. To determine these effects, the inventors performed RNA-seq on total RNA isolated from E. coli cells transformed with either one of the four STARs or the no-antisense control plasmid. It should be noted that E. coli strain K12 MG1655 was used, in which the inventors showed our STAR antisenses to be functional. Differential gene expression analysis between a specific STAR antisense condition and our no-antisense control showed that there were global changes in gene expression due to STAR antisense expression, although the majority of genes are unaffected. The inventors also found this to be true for the best ribA STAR antisense. As each STAR antisense seems to behave similarly, these observed changes in gene expression could be due to a general response to the presence of a highly expressed RNA.

### Example 9. Applying STARs to Construct Novel RNA-Only Logic Gates

The inventors aimed to construct new RNA-only transcriptional logic gates that were previously unattainable owing to the lack of sRNA activators. Genetic logic gates are necessary network elements for constructing circuits that integrate signals and process information to control cellular behavior. However, the only synthetic RNA-mediated transcriptional logic gates that have been demonstrated are NOR gates, which only allow gene expression when none of the gate inputs are present. The inventors therefore sought to construct two new RNA-only transcriptional logic gates that combined both RNA transcriptional activators and attenuators: an A AND B gate (FIG. 9A) and an A AND NOT B gate (FIG. 9B). These logic gates were constructed by transcriptionally fusing STAR target sense and attenuator sequences in series and were tested against all possible input combinations of antisense sRNAs. This characterization revealed that both the A AND B and A AND NOT B gates were functional; the AND gate was only ON when both inputs were present, whereas the A AND NOT B was in the ON state when only the A input was present. These results provided further evidence that STARs act on the transcriptional level and demonstrated that STARs can be used within more sophisticated RNA genetic circuitry devices.

### Example 10. Genomic Integration of STAR Activating Genetic Constructs in Bacillus

The inventors designed a sense RNA and an activating STAR to regulate expression of a target gene in Bacillus subtilis strain 168. To do this, the inventors designed a plasmid with a sense RNA upstream of an RBS and a GFP gene, and downstream of a constitutive promoter. The inventors further designed a STAR construct as a constitutive promoter followed by the antisense sequence followed by its own terminator. The DNA constructs are integrated into the genome of B. subtilis at the amyE (alpha-amylase) gene as an initial test of the design. Following genomic integration, target gene expression can be determined by measuring fluorescence in the presence and absence of the STAR.

### Example 11. STAR Regulation in Eukaryotic Cells

Sense RNA and STAR constructs are designed for transformation of a eukaryotic cell in vitro. The eukaryotic cell is a yeast cell or a plant, insect, or mammalian cell.

The sense RNA construct is upstream of a eukaryotic translation initiation sequence and a gene to be regulated, and downstream of a bacteriophage promoter, such as a promoter recognized by T7 phage polymerase.

The STAR is downstream of a bacteriophage promoter, followed by its own terminator.

The bacteriophage RNA polymerase sequence is placed downstream of a eukaryotic promoter and eukaryotic translation initiation sequences. The RNA polymerase gene is followed by eukaryotic transcription and translation termination sequences.

The bacteriophage phage RNA polymerase is expressed by the host cell and proceeds to transcribe the sense RNA and the STAR to enact regulation. If gene to be regulated is transcribed then the eukaryotic translation machinery will translates the gene.

### Example 12. STAR Regulation Improves Existing Technologies

STAR sense/antisense regulation of enzymatic pathways can be used to improve metabolic pathways for enzyme expression and strain engineering (FIG. 10A).

### Example 13. Sense/Antisense Sequences Validate Design Principles

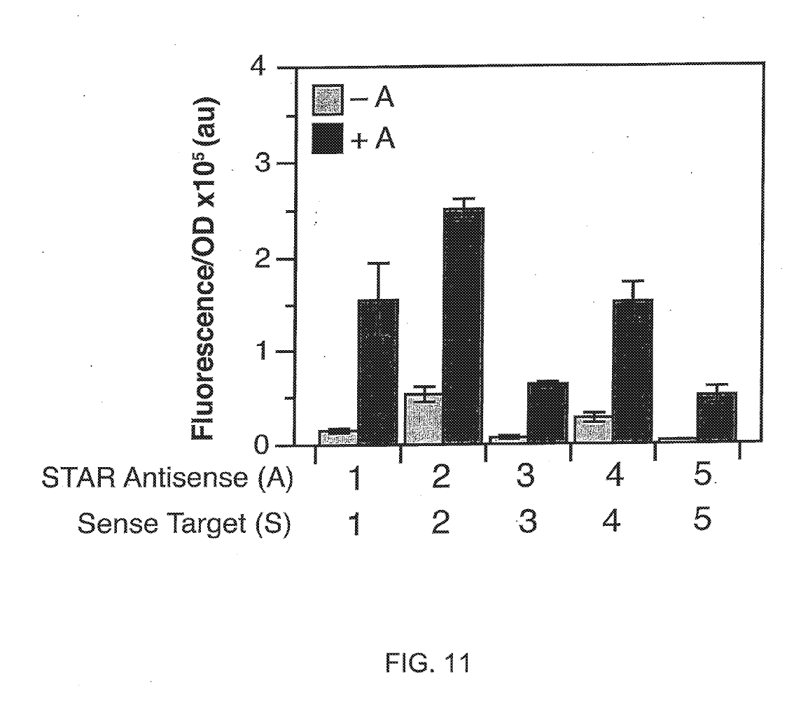

Sense/antisense sequences designed according to the disclosed methods function to activate transcription (FIG. 11). Each combination of STAR/sense sequence provides increased activation of transcription of the target gene, thus validating the design principles.

TABLE 2
Sense Sequences

| DESCRIPTION | SEQUENCE ID NO. |
|---|---|
| Anti-anti.S1 | 38 |
| Anti-anti.S2 | 39 |
| Anti-anti.S3 | 40 |
| Anti-anti.S4 | 41 |
| T181.S1 | 1 |
| T181.S2 | 2 |
| T181.S3 | 3 |
| T181.S4 | 4 |
| T181.S5 | 5 |
| T181.S6 | 6 |
| T181.S7 | 7 |
| T181.S8 | 8 |
| T181.S9 | 9 |
| T181.S10 | 10 |
| T181.S11 | 11 |
| AD1.S1 | 12 |
| IP501.S1 | 13 |
| CF10.S1 | 14 |
| AD1.S2 | 15 |
| AD1.S3 | 16 |
| AD1.S4 | 17 |
| AD1.S5 | 18 |
| AD1.S6 | 19 |
| AD1.S7 | 20 |
| AD1.S7 | 21 |
| metH.S1 | 22 |
| xpt.S1 | 23 |
| pbuE.S1 | 24 |
| pbuE.S2 | 25 |
| pbuE.S3 | 26 |
| pbuE.S4 | 27 |
| pbuE.S5 | 28 |
| pbuE.S6 | 29 |
| pT181.H1 | 30 |
| Fusion 6 | 31 |
| Fusion 4m1 | 32 |
| Fusion 4 | 33 |
| Cst.S1 | 34 |
| gpt.S1 | 35 |
| rmf.S1 | 36 |
| ribA.S1 | 37 |
| De novo S1 | 105 |
| De novo S2 | 106 |
| De novo S3 | 107 |
| De novo S4 | 108 |
| De novo S5 | 109 |
| De novo S6 | 110 |
| De novo S7 | 111 |
| De novo S8 | 112 |
| De novo S9 | 113 |
| De novo S10 | 114 |

TABLE 3
ANTISENSE SEQUENCES

| DESCRIPTION | SEQUENCE ID NO. |
|---|---|
| Anti.anti.A1 | 84 |
| Anti.anti.A2 | 85 |
| Anti.anti.A3 | 86 |
| Anti.anti.A4 | 87 |
| T181.A1 | 42 |
| T181.A2 | 43 |
| T181.A3 | 44 |
| T181.A4 | 45 |
| T181.A5 | 46 |
| T181.A6 | 47 |
| T181.A7 | 48 |
| T181.A8 | 49 |
| T181.S9 | 50 |
| T181.S10 | 51 |
| AD1.A1 | 52 |
| IP501.A1 | 53 |
| CF10.A1 | 54 |
| AD1.A2 | 55 |
| AD1.A3 | 56 |
| AD1.A4 | 57 |
| AD1.A5 | 58 |
| AD1.A6 | 59 |
| AD1.A7 | 60 |
| meth.A1 | 61 |
| Xpt.A1 | 62 |
| pbuE.A1 | 63 |
| pbuE.A2 | 64 |
| pbuE.A3 | 65 |
| pbuE.A4 | 66 |
| pbuE.A5 | 67 |
| pbuE.A6 | 68 |
| pT181.H1 | 69 |
| Fusion 6 | 70 |
| Fusion 4m1 | 71 |
| Fusion 4 | 72 |
| cst.A1 | 73 |
| gpt.A1 | 74 |
| rmf.A1 | 75 |
| ribA.A1 | 76 |
| ribA.A2 | 77 |
| ribA.A3 | 78 |
| ribA.A4 | 79 |
| ribA.A5 | 80 |
| ribA.A6 | 81 |
| ribA.A7 | 82 |
| ribA.A8 | 83 |
| De novo A1 | 110 |
| De novo A2 | 111 |
| De novo A3 | 112 |
| De novo A4 | 113 |
| De novo A5 | 114 |

TABLE 4
Sample Plasmid Sequences

| DESCRIPTION | SEQUENCE ID NO. |
|---|---|
| Sense plasmid: T181.S1 (EcoRI-J23119- | 91 |
| Sense-RBS-SFGFP-TrrnB-PstI-CmR- | |
| p15A origin) | |
| Antisense plasmid: T181.A1 (EcoRI- | 97 |
| J23119-Antisense-t500-PstI-ColE1 | |
| origin-AmpR) | |
| A AND B sense plasmid (EcoRI-J23119- | 101 |
| Anti-anti.S4-AD1.S5-RBS-SFGFP- | |
| TrrnB-PstI-CmR-p15A origin) | |
| A AND NOT B sense plasmid (EcoRI- | 102 |
| J23119-pT181.H1-AD1.S5-RBS- | |
| SFGFP-TrrnB-PstI-CmR-p15A origin) | |
| A AND B Antisense plasmid (EcoRI- | 103 |
| J23119-Anti.anti.A4-t500-buffer region- | |
| J23119-AD1.A5-t500-ColE1 origin- | |
| AmpR) | |
| A AND NOT B Antisense plasmid (EcoRI- | 115 |
| J23119-pT181.H1-TrrnB-buffer region- | |
| J23119-AD1.A5-t500-PstI-ColE1 | |
| origin-AmpR) | |
| J23119 promoter | 92 |
| SFGFP | 93 |
| TrrnB transcription terminator | 94 |
| Chloramphenicol resistance | 95 |
| P15A origin of replication | 96 |
| t500 transcription terminator | 98 |
| ColE1 origin of replication | 99 |
| Ampicillin resistance | 100 |
| Buffer sequence between STAR antisense | 104 |
| coding regions | |

TABLE 5
PRIMERS

| DESCRIPTION | SEQUENCE ID NO. |
|---|---|
| RT.SFGFP | 88 |
| SFGFP.Fwd | 89 |
| SFGFP.Rev | 90 |

What is claimed is:

1. A composition for transcriptional regulation of a target gene comprising:
a. a sense genetic construct comprising, from 5′ to 3′: a promoter sequence; a terminator sequence encoding a ribonucleic acid (RNA) terminator stem-loop, said terminator sequence comprising a 5′ terminator stem sequence and a 3′ terminator stem sequence that are substantially complementary to each other; and a sequence encoding a poly-uracil RNA sequence immediately 3′ of the terminator sequence; and
b. an antisense construct, selected from (i) an antisense activating RNA with substantial complementary to at least a portion of the 5′ terminator stem sequence of the sense RNA, or (ii) an antisense genetic construct encoding an antisense activating RNA, said antisense genetic construct comprising, from 5′ to 3′: a promoter sequence and a sequence encoding an antisense activating RNA with substantial complementary to at least a portion of the 5′ terminator stem sequence of the sense RNA.

2. The composition of claim 1, wherein said terminator sequence is 10 to 300 nucleotides in length.

3. The composition of claim 1, wherein the 5′ terminator stem sequence is 4 to 40 nucleotides in length.

4. The composition of claim 1, wherein the 5′ terminator stem sequence has a G-C content of at least 50%.

5. The composition of claim 1, wherein the poly-uracil sequence of said sense genetic construct is 5-12 nucleotides in length.

6. The composition of claim 1, wherein the poly-uracil sequence of said sense genetic construct is composed of at least 50% uracils.

7. The composition of claim 1, wherein said sense terminator sequence has at least 85% identity to a nucleic acid sequence selected from SEQ ID NOS: 1-41.

8. The composition of claim 1, wherein said sense genetic construct comprises a constitutive, inducible, or tissue-specific promoter.

9. The composition of claim 1, wherein the sense genetic construct does not contain a sequence between the promoter and the 5′ terminator stem sequence with substantial complementarity to the 5′ terminator stem sequence.

10. The composition of claim 1, wherein said sense genetic construct comprises at least two terminator sequences in tandem.

11. The composition of claim 10, further comprising at least two antisense constructs.

12. The composition of claim 1, wherein said antisense activating RNA is 5 to 300 nucleotides in length.

13. The composition of claim 1, wherein said antisense activating RNA sequence has at least 85% identity to a nucleic acid sequence selected from SEQ ID NOS: 42-87.

14. The composition of claim 1, wherein the antisense construct is an antisense genetic construct encoding said antisense activating RNA.

15. The composition of claim 14, wherein said antisense genetic construct comprises a transcriptional termination sequence after the antisense activating RNA sequence.

16. The composition of claim 14, wherein the sense genetic construct and the antisense genetic construct are on separate vectors.

17. The composition of claim 14, wherein said antisense genetic construct comprises a constitutive, inducible, or tissue-specific promoter.

18. The composition of claim 14, wherein the sense genetic construct and the antisense genetic construct have different promoters.

19. The composition of claim 1, further comprising an RNA polymerase.

20. The composition of claim 19, wherein the RNA polymerase is selected from T7, T5, or T3 bacteriophage polymerase, SP6 bacteriophage polymerase, U6 bacteriophage polymerase, H1 human polymerase, or Bst bacterial polymerase.

21. A method of regulating expression of a gene of interest, comprising placing the gene of interest under control of the composition of claim 1.

22. The method of claim 21, wherein the sense genetic construct is inserted into a genomic sequence upstream of a gene of interest.

23. The method of claim 21, wherein in the absence of the antisense activating RNA, transcription of the gene of interest is repressed.

24. The method of claim 21, wherein the antisense activating RNA activates transcription of the gene of interest.

25. The method of claim 21, wherein said composition is introduced into a prokaryotic cell.

26. The method of claim 21, wherein said composition is introduced into a eukaryotic cell in vitro.

27. The method of claim 21, wherein said gene of interest is endogenous to said cell.

28. The method of claim 21, wherein said gene of interest is not endogenous to said cell.

29. The method of claim 21, wherein said composition is introduced into a host cell for an industrial fermentation, biofuel production, or recombinant protein production process.

30. The method of claim 21, wherein said sense genetic construct mimics a riboswitch, aptazyme, or a sequence that can be recognized by antisense repressor RNA molecules.

31. A method of increasing the transcription of a target gene, comprising introducing the composition of claim 1 into a host cell so that said sense genetic construct is in operable linkage with said target gene and expression of said antisense activating construct increases transcription of said target gene.

32. The method of claim 31, wherein said host cell is a eukaryotic cell.

33. An in vitro transcription-translation system comprising a cell extract; a reporter genetic construct comprising a sense genetic construct in operable linkage to a detectable reporter gene; and an activating antisense construct.


SEQUENCE LISTING (115 sequences)

SEQ ID NO: 1 - DNA, 139 bp, Artificial Sequence
  Synthetic Oligonucleotide
cgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 2 - DNA, 259 bp, Artificial Sequence
  Synthetic Oligonucleotide
ctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 3 - DNA, 231 bp, Artificial Sequence
  Synthetic Oligonucleotide
caaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 4 - DNA, 148 bp, Artificial Sequence
  Synthetic Oligonucleotide
ttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 5 - DNA, 152 bp, Artificial Sequence
  Synthetic Oligonucleotide
ttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 6 - DNA, 158 bp, Artificial Sequence
  Synthetic Oligonucleotide
atatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 7 - DNA, 168 bp, Artificial Sequence
  Synthetic Oligonucleotide
acataaagatatatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 8 - DNA, 178 bp, Artificial Sequence
  Synthetic Oligonucleotide
atttaaatatacataaagatatatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 9 - DNA, 188 bp, Artificial Sequence
  Synthetic Oligonucleotide
attaaacgatatttaaatatacataaagatatatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 10 - DNA, 198 bp, Artificial Sequence
  Synthetic Oligonucleotide
atatttagatattaaacgatatttaaatatacataaagatatatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 11 - DNA, 208 bp, Artificial Sequence
  Synthetic Oligonucleotide
ataatcttgtatatttagatattaaacgatatttaaatatacataaagatatatatttgggtgagcgattccttaaacgaaattgagattaaggagtcgctcttttttttttatgtataaaaacaatcatgcaaatcattcaaatcatttggaaaatcacgatttagacaatttttctaaaaccggctactctaatagccggttgtaa

SEQ ID NO: 12 - DNA, 58 bp, Artificial Sequence
  Synthetic Oligonucleotide
aatgttggagcagcggggaatgtatacagttcatgtatatattccccgcttttttttt

SEQ ID NO: 13 - DNA, 59 bp, Artificial Sequence
  Synthetic Oligonucleotide
aagtctttaagaagataccaggcaataattaagaaaaacttagttgattgccttttttt

SEQ ID NO: 14 - DNA, 56 bp, Artificial Sequence
  Synthetic Oligonucleotide
aatgttgagcagcggggaatgtatacagttcatgtatatgttccccgctttttttt

SEQ ID NO: 15 - DNA, 63 bp, Artificial Sequence
  Synthetic Oligonucleotide
gtataaatgttggagcagcggggaatgtatacagttcatgtatatattccccgcttttttttt

SEQ ID NO: 16 - DNA, 73 bp, Artificial Sequence
  Synthetic Oligonucleotide
ttaattagttgtataaatgttggagcagcggggaatgtatacagttcatgtatatattccccgcttttttttt

SEQ ID NO: 17 - DNA, 83 bp, Artificial Sequence
  Synthetic Oligonucleotide
gtgaattgttttaattagttgtataaatgttggagcagcggggaatgtatacagttcatgtatatattccccgcttttttttt

SEQ ID NO: 18 - DNA, 93 bp, Artificial Sequence
  Synthetic Oligonucleotide
agtttttacagtgaattgttttaattagttgtataaatgttggagcagcggggaatgtatacagttcatgtatatattccccgcttttttttt

SEQ ID NO: 19 - DNA, 103 bp, Artificial Sequence
  Synthetic Oligonucleotide
gtctaggaaaagtttttacagtgaattgttttaattagttgtataaatgttggagcagcggggaatgtatacagttcatgtatatattccccgcttttttttt

SEQ ID NO: 20 - DNA, 113 bp, Artificial Sequence
  Synthetic Oligonucleotide
tcctttatttgtctaggaaaagtttttacagtgaattgttttaattagttgtataaatgttggagcagcggggaatgtatacagttcatgtatatattccccgcttttttttt

SEQ ID NO: 21 - DNA, 113 bp, Artificial Sequence; identical to SEQ ID NO: 20
  Synthetic Oligonucleotide

SEQ ID NO: 22 - DNA, 56 bp, Artificial Sequence
  Synthetic Oligonucleotide
cggcgctcgcaaacccgcgttttccttgccccggtttgcggcgccgtttttttttt

SEQ ID NO: 23 - DNA, 58 bp, Artificial Sequence
  Synthetic Oligonucleotide
cggttttttgtgatatcagcattgcttgctctttatttgagcgggcaatgcttttttt

SEQ ID NO: 24 - DNA, 73 bp, Artificial Sequence
  Synthetic Oligonucleotide
gtgtctaccaggaaccgtaaaatcctgattacaaaatttgtttatgacattttttgtaatcaggatttttttt

SEQ ID NO: 25 - DNA, 80 bp, Artificial Sequence
  Synthetic Oligonucleotide
tttgagggtgtctaccaggaaccgtaaaatcctgattacaaaatttgtttatgacattttttgtaatcaggatttttttt

SEQ ID NO: 26 - DNA, 90 bp, Artificial Sequence
  Synthetic Oligonucleotide
aataatatggtttgagggtgtctaccaggaaccgtaaaatcctgattacaaaatttgtttatgacattttttgtaatcaggatttttttt

SEQ ID NO: 27 - DNA, 100 bp, Artificial Sequence
  Synthetic Oligonucleotide
gtataacctcaataatatggtttgagggtgtctaccaggaaccgtaaaatcctgattacaaaatttgtttatgacattttttgtaatcaggatttttttt

SEQ ID NO: 28 - DNA, 110 bp, Artificial Sequence
  Synthetic Oligonucleotide
attatcacttgtataacctcaataatatggtttgagggtgtctaccaggaaccgtaaaatcctgattacaaaatttgtttatgacattttttgtaatcaggatttttttt

SEQ ID NO: 29 - DNA, 120 bp, Artificial Sequence
  Synthetic Oligonucleotide
ttaaatagctattatcacttgtataacctcaataatatggtttgagggtgtctaccaggaaccgtaaaatcctgattacaaaatttgtttatgacattttttgtaatcaggatttttttt

SEQ ID NO: 30 - DNA, 287 bp, Artificial Sequence
  Synthetic Oligonucleotide
aacaaaataaaaaggagtcgctctgtccctcgccaaagttgcagaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 31 - DNA, 292 bp, Artificial Sequence
  Synthetic Oligonucleotide
aacaaaataaaaaggagtcgctcacttacgaacttggcggaacgacgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 32 - DNA, 293 bp, Artificial Sequence
  Synthetic Oligonucleotide
aacaaaataaaaaggagtcgctcacgttcatgattggcgtcaacgatgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 33 - DNA, 293 bp, Artificial Sequence
  Synthetic Oligonecleotide
aacaaaataaaaaggagtcgctcacgttcaactttggcgagtacgatgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 34 - DNA, 66 bp, Artificial Sequence
  Synthetic Oligonucleotide
ttagtgcccagggttccctctcaccctaaccctctccccggtggggcgaggggactgaccgagcgc

SEQ ID NO: 35 - DNA, 153 bp, Artificial Sequence
  Synthetic Oligonucleotide
gtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggc

SEQ ID NO: 36 - DNA, 152 bp, Artificial Sequence
  Synthetic Oligonucleotide
acgctcaaaagaaatgtgtccctatcagacgctgaatcaaaggtcacaatggctgggaggctggcgagaagccatggcggacagggtagtaatggcctgattctgtctctttaaaaagaaacctccgcattgcggaggtttcgccttttgat

SEQ ID NO: 37 - DNA, 168 bp, Artificial Sequence
  Synthetic Oligonucleotide
tattgttgaacgcgtaccattgattgtaggtcgtaaccccaataacgaacattatctcgataccaaagccgagaaaatgggccatttgctgaacaaataaccctcttgcattgtgtaattcatttgcttgccggaagcaaaataaccggcaagcaaatagttgttact

SEQ ID NO: 38 - DNA, 322 bp, Artificial Sequence
  Synthetic Oligonucleotide
aacaaaatcgactcctttttcttacgaacttggcggaacgacgaaaaaggagtcgctcacgccctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 39 - DNA, 319 bp, Artificial Sequence
  Synthetic Oligonucleotide
aacaaaatcgactcctttttttcatgattggcgtcaacgaaaaaaggagtcgctcacgccctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 40 - DNA, 336 bp, Artificial Sequence
  Synthetic Oligonucleotide
aacaaaatcgactccttttttctgattattgatttttcgcgaaaccatttaatcataaaaaaggagtcgctcacgccctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 41 - DNA, 320 bp, Artificial Sequence
  Synthetic Oligonucleotide
aacaaaatcgactcctttttcctatgtctagtccacatcagaaaaaggagtcgctcacgccctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaa

SEQ ID NO: 42 - DNA, 17 bp, Artificial Sequence
  Synthetic Oligonucleotide
aaggagtcgctcacgcc

SEQ ID NO: 43 - DNA, 28 bp, Artificial Sequence
  Synthetic Oligonucleotide
aacaaaataaaaaggagtcgctcacgcc

SEQ ID NO: 44 - DNA, 35 bp, Artificial Sequence
  Synthetic Oligonucleotide
aacaaaataaacgtttaaggaatcgctcacccaaa

SEQ ID NO: 45 - DNA, 35 bp, Artificial Sequence
  Synthetic Oligonucleotide
aacaaaataaagcaataaggaatcgctcacccaaa

SEQ ID NO: 46 - DNA, 40 bp, Artificial Sequence
  Synthetic Oligonucleotide
aacaaaataaagcaataaggaatcgctcacccaaatatat

SEQ ID NO: 47 - DNA, 50 bp, Artificial Sequence
  Synthetic Oligonucleotide
aacaaaataaagcaataaggaatcgctcacccaaatatatatctttatgt

SEQ ID NO: 48 - DNA, 60 bp, Artificial Sequence
  Synthetic Oligonucleotide
aacaaaataaagcaataaggaatcgctcacccaaatatatatctttatgtatatttaaat

SEQ ID NO: 49 - DNA, 70 bp, Artificial Sequence
  Synthetic Oligonucleotide
aacaaaataaagcaataaggaatcgctcacccaaatatatatctttatgtatatttaaatatcgtttaat

SEQ ID NO: 50 - DNA, 80 bp, Artificial Sequence
  Synthetic Oligonucleotide
aacaaaataaagcaataaggaatcgctcacccaaatatatatctttatgtatatttaaatatcgtttaatatctaaatat

SEQ ID NO: 51 - DNA, 90 bp, Artificial Sequence
  Synthetic Oligonucleotide
aacaaaataaagcaataaggaatcgctcacccaaatatatatctttatgtatatttaaatatcgtttaatatctaaatatacaagattat

SEQ ID NO: 52 - DNA, 33 bp, Artificial Sequence
  Synthetic Oligonucleotide
tgaactgtatacattccccgctgctccaacatt

SEQ ID NO: 53 - DNA, 38 bp, Artificial Sequence
  Synthetic Oligonucleotide
tttttcttaattattgcctggtatcttcttaaagactt

SEQ ID NO: 54 - DNA, 32 bp, Artificial Sequence
  Synthetic Oligonucleotide
tgaactgtatacattccccgctgctcaacatt

SEQ ID NO: 55 - DNA, 32 bp, Artificial Sequence; identical to SEQ ID NO: 54
  Synthetic Oligonucleotide

SEQ ID NO: 56 - DNA, 48 bp, Artificial Sequence
  Synthetic Oligonucleotide
tgaactgtatacattccccgctgctccaacatttatacaactaattaa

SEQ ID NO: 57 - DNA, 58 bp, Artificial Sequence
  Synthetic Oligonucleotide
tgaactgtatacattccccgctgctccaacatttatacaactaattaaaacaattcac

SEQ ID NO: 58 - DNA, 68 bp, Artificial Sequence
  Synthetic Oligonucleotide
tgaactgtatacattccccgctgctccaacatttatacaactaattaaaacaattcactgtaaaaact

SEQ ID NO: 59 - DNA, 78 bp, Artificial Sequence
  Synthetic Oligonucleotide
tgaactgtatacattccccgctgctccaacatttatacaactaattaaaacaattcactgtaaaaacttttcctagac

SEQ ID NO: 60 - DNA, 88 bp, Artificial Sequence
  Synthetic Oligonucleotide
tgaactgtatacattccccgctgctccaacatttatacaactaattaaaacaattcactgtaaaaacttttcctagacaaataaagga

SEQ ID NO: 61 - DNA, 26 bp, Artificial Sequence
  Synthetic Oligonucleotide
aggaaaacgcgggtttgcgagcgccg

SEQ ID NO: 62 - DNA, 38 bp, Artificial Sequence
  Synthetic Oligonucleotide
aaataaagagcaagcaatgctgatatcacaaaaaaccg

SEQ ID NO: 63 - DNA, 45 bp, Artificial Sequence
  Synthetic Oligonucleotide
ataaacaaattttgtaatcaggattttacggttcctggtagacac

SEQ ID NO: 64 - DNA, 52 bp, Artificial Sequence
  Synthetic Oligonucleotide
ataaacaaattttgtaatcaggattttacggttcctggtagacaccctcaaa

SEQ ID NO: 65 - DNA, 62 bp, Artificial Sequence
  Synthetic Oligonucleotide
ataaacaaattttgtaatcaggattttacggttcctggtagacaccctcaaaccatattatt

SEQ ID NO: 66 - DNA, 72 bp, Artificial Sequence
  Synthetic Oligonucleotide
ataaacaaattttgtaatcaggattttacggttcctggtagacaccctcaaaccatattattgaggttatac

SEQ ID NO: 67 - DNA, 82 bp, Artificial Sequence
  Synthetic Oligonucleotide
ataaacaaattttgtaatcaggattttacggttcctggtagacaccctcaaaccatattattgaggttatacaagtgataat

SEQ ID NO: 68 - DNA, 92 bp, Artificial Sequence
  Synthetic Oligonucleotide
ataaacaaattttgtaatcaggattttacggttcctggtagacaccctcaaaccatattattgaggttatacaagtgataatagctatttaa

SEQ ID NO: 69 - DNA, 91 bp, Artificial Sequence
  Synthetic Oligonucleotide
atacaagattataaaaacaactcagtgtttttttctttgaatgatgtcgttctgcaactttggcgagggacagagcgactcctttttattt

SEQ ID NO: 70 - DNA, 96 bp, Artificial Sequence
  Synthetic Oligonucleotide
atacaagattataaaaacaactcagtgtttttttctttgaatgatgtcgttcacgtcgttccgccaagttcgtaagtgagcgactcctttttattt

SEQ ID NO: 71 - DNA, 97 bp, Artificial Sequence
  Synthetic Oligonucleotide
atacaagattataaaaacaactcagtgtttttttctttgaatgatgtcgttcacatcgttgacgccaatcatgaacgtgagcgactcctttttattt

SEQ ID NO: 72 - DNA, 97 bp, Artificial Sequence
  Synthetic Oligonucleotide
atacaagattataaaaacaactcagtgtttttttctttgaatgatgtcgttcacatcgtactcgccaaagttgaacgtgagcgactcctttttattt

SEQ ID NO: 73 - DNA, 39 bp, Artificial Sequence
  Synthetic Oligonucleotide
ggggagagggttagggtgagagggaaccctgggcactaa

SEQ ID NO: 74 - DNA, 77 bp, Artificial Sequence
  Synthetic Oligonucleotide
agtgccaggcgttgaaaagattagcgaccggagattggcgggacgaatacgacgcccatatcccacggctgttcaat

SEQ ID NO: 75 - DNA, 43 bp, Artificial Sequence
  Synthetic Oligonucleotide
aatgcggaggtttctttttaaagagacagaatcaggccattac

SEQ ID NO: 76 - DNA, 34 bp, Artificial Sequence
  Synthetic Oligonucleotide
ttattttgcttccggcaagcaaatgaattacaca

SEQ ID NO: 77 - DNA, 44 bp, Artificial Sequence
  Synthetic Oligonucleotide
ttattttgcttccggcaagcaaatgaattacacaatgcaagagg

SEQ ID NO: 78 - DNA, 49 bp, Artificial Sequence
  Synthetic Oligonucleotide
ttattttgcttccggcaagcaaatgaattacacaatgcaagagggttat

SEQ ID NO: 79 - DNA, 54 bp, Artificial Sequence
  Synthetic Oligonucleotide
ttattttgcttccggcaagcaaatgaattacacaatgcaagagggttatttgtt

SEQ ID NO: 80 - DNA, 58 bp, Artificial Sequence
  Synthetic Oligonucleotide
ttattttgcttccggcaagcaaatgaattacacaatgcaagagggttatttgttcagc

SEQ ID NO: 81 - DNA, 64 bp, Artificial Sequence
  Synthetic Oligonucleotide
ttattttgcttccggcaagcaaatgaattacacaatgcaagagggttatttgttcagcaaatgg

SEQ ID NO: 82 - DNA, 71 bp, Artificial Sequence
  Synthetic Oligonucleotide
ttattttgcttccggcaagcaaatgaattacacaatgcaagagggttatttgttcagcaaatggcccattt

SEQ ID NO: 83 - DNA, 84 bp, Artificial Sequence
  Synthetic Oligonucleotide
ttattttgcttccggcaagcaaatgaattacacaatgcaagagggttatttgttcagcaaatggcccattttctcggctttggt

SEQ ID NO: 84 - DNA, 55 bp, Artificial Sequence
  Synthetic Oligonucleotide
tgagcgactcctttttcgtcgttccgccaagttcgtaagaaaaaggagtcgattt

SEQ ID NO: 85 - DNA, 53 bp, Artificial Sequence
  Synthetic Oligonucleotide
gtgagcgactccttttttcgttgacgccaatcatgaaaaaaaggagtcgattt

SEQ ID NO: 86 - DNA, 36 bp, Artificial Sequence
  Synthetic Oligonucleotide
gcgaaaaatcaataatcagaaaaaaggagtcgattt

SEQ ID NO: 87 - DNA, 29 bp, Artificial Sequence
  Synthetic Oligonucleotide
gactagacataggaaaaaggagtcgattt

SEQ ID NO: 88 - DNA, 22 bp, Artificial Sequence
  Synthetic Oligonucleotide
ttatttgtagagctcatccatg

SEQ ID NO: 89 - DNA, 21 bp, Artificial Sequence
  Sythetic Oligonucleotide
cactggagttgtcccaattct

SEQ ID NO: 90 - DNA, 21 bp, Artificial Sequence
  Synthetic Oligonucleotide
tccgtttgtagcatcaccttc

SEQ ID NO: 91 - DNA, 3471 bp, Artificial Sequence
  Synthetic Oligonucleotide
gaattctaaagatctttgacagctagctcagtcctaggtataatactagtcgattccttaaacgaaattgagattaaggagtcgctcttttttatgtataaaaacaatcatgcaaatcattcaaatcatttggaaaatcacgatttagacaatttttctaaaaccggctactctaatagccggttgtaaggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttacactttatgcttccggct

SEQ ID NO: 92 - DNA, 35 bp, Artificial Sequence
  Synthetic Oligonucleotide
ttgacagctagctcagtcctaggtataatactagt

SEQ ID NO: 93 - DNA, 717 bp, Artificial Sequence
  Synthetic Oligonucleotide
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 94 - DNA, 368 bp, Artificial Sequence
  Synthetic Oligonucleotide
gaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaact

SEQ ID NO: 95 - DNA, 864 bp, Artificial Sequence
  Synthetic Oligonucleotide
ggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttatt

SEQ ID NO: 96 - DNA, 845 bp, Artificial Sequence
  Synthetic Oligonucleotide
gcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatattt

SEQ ID NO: 97 - DNA, 2166 bp, Artificial Sequence
  Synthetic Oligonucleotide
gaattctaaagatctttgacagctagctcagtcctaggtataatactagtaaggagtcgctcacgccggatctcaaagcccgccgaaaggcgggcttttttttggatccttactcgagtctagactgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctg

SEQ ID NO: 98 - DNA, 30 bp, Artificial Sequence
  Synthetic Oligonucleotide
caaagcccgccgaaaggcgggctttttttt

SEQ ID NO: 99 - DNA, 683 bp, Artificial Sequence
  Synthetic Oligonucleotide
ggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 100 - DNA, 660 bp, Artificial Sequence
  Synthetic Oligonucleotide
ccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcat

SEQ ID NO: 101 - DNA, 3751 bp, Artificial Sequence
  Synthetic Oligonucleotide
gaattctaaagatctttgacagctagctcagtcctaggtataatactagtaacaaaatcgactcctttttcctatgtctagtccacatcagaaaaaggagtcgctcacgccctgaccaaagtttgtgaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaaggatctagtttttacagtgaattgttttaattagttgtataaatgttggagcagcggggaatgtatacagttcatgtatatattccccgctttttttttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttacactttatgcttccggct

SEQ ID NO: 102 - DNA, 3718 bp, Artificial Sequence
  Synthetic Oligonucleotide
gaattctaaagatctttgacagctagctcagtcctaggtataatactagtaacaaaataaaaaggagtcgctctgtccctcgccaaagttgcagaacgacatcattcaaagaaaaaaacactgagttgtttttataatcttgtatatttagatattaaacgatatttaaatatacataaagatatatatttgggtgagcgattccttaaacgaaattgagattaaggagtcgctcttttttatgtataaaaacaatcatgcaaatcattcaaatcatttggaaaatcacgatttagacaatttttctaaaaccggctactctaatagccggttgtaaggatctagtttttacagtgaattgttttaattagttgtataaatgttggagcagcggggaatgtatacagttcatgtatatattccccgctttttttttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttacactttatgcttccggct

SEQ ID NO: 103 - DNA, 2563 bp, Artificial Sequence
  Synthetic Oligonucleotide
gaattctaaagatctttgacagctagctcagtcctaggtataatactagtgactagacataggaaaaaggagtcgatttcaaagcccgccgaaaggcgggcttttttttggatccttactcgagtctagactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctggaattctaaagatctttgacagctagctcagtcctaggtataatactagttgaactgtatacattccccgctgctccaacatttatacaactaattaaaacaattcactgtaaaaactggatctcaaagcccgccgaaaggcgggcttttttttgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctg

SEQ ID NO: 104 - DNA, 275 bp, Artificial Sequence
  Synthetic Oligonucleotide
ggatccttactcgagtctagactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctggaattctaaagatct

SEQ ID NO: 105 - DNA, 69 bp, Artificial Sequence
  Synthetic Oligonucleotide
gggacctctacttactctcactcttacgtatgcatagttcatatgcacttgtaagagtgtttttttttt

SEQ ID NO: 106 - DNA, 69 bp, Artificial Sequence
  Synthetic Oligoneucleotide
gggcttactactttgacacctgattctgtaacgatagttcatatcgtcagagaatcaggtttttttttt

SEQ ID NO: 107 - DNA, 69 bp, Artificial Sequence
  Synthetic Oligonucleotide
gggcgaatagaaatgaaggctagtgtcgtagtcatagttcatatgacttggacactagctttttttttt

SEQ ID NO: 108 - DNA, 70 bp, Artificial Sequence
  Synthetic Oligonucleotide
gggcaatttcgtatatgttcgtctttggtattcatagttcatatgaagtccaaagacgaatttttttttt

SEQ ID NO: 109 - DNA, 69 bp, Artificial Sequence
  Synthetic Oligonucleotide
gggcgaatagaaatgaaggctagtgtcgtagtcatagttcatatgactacgacactagctttttttttt

SEQ ID NO: 110 - DNA, 65 bp, Artificial Sequence
  Synthetic Oligonucleotide
gggactgagctgctatcacgcagctcagtagagcacatgtaagagtgagagtaagtagaggtaga

SEQ ID NO: 111 - DNA, 65 bp, Artificial Sequence
  Synthetic Oligonucleotide
gggtcaattacccgtggtagggtaattgaaagcgtcatagaatcaggtgtcaaagtagtaagtag

SEQ ID NO: 112 - DNA, 65 bp, Artificial Sequence
  Synthetic Oligonucleotide
gggactccacgccgaccgtggcgtggagtaaagaccatgacactagccttcatttctattcgatt

SEQ ID NO: 113 - DNA, 65 bp, Artificial Sequence
  Synthetic Oligonucleotide
gggcgacgatgccaggtatggcatcgtcggacgaacatcaaagacgaacatatacgaaattgaaa

SEQ ID NO: 114 - DNA, 65 bp, Artificial Sequence
  Synthetic Oligonucleotide
gggactccacgccgaccgtggcgtggagtaaagactacgacactagccttcatttctattcgatt

SEQ ID NO: 115 - DNA, 2413 bp, Artificial Sequence
  Synthetic Oligonucleotide
gaattctaaagatctttgacagctagctcagtcctaggtataatactagtatacaagattataaaaacaactcagtgtttttttctttgaatgatgtcgttctgcaactttggcgagggacagagcgactcctttttatttggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcaggcttcctcgctcactgactcgctgcgctcggtcgttcggctgcggcgagcggtatcagctcactcaaaggcgggcttttttttccttagctttcgctaaggatgatttctggaattctaaagatctttgacagctagctcagtcctaggtataatactagttgaactgtatacattccccgctgctccaacatttatacaactaattaaaacaattcactgtaaaaactggatctcaaagcccgccgaaaggcgggcttttttttggatccttactcgagtctagactgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagca